(19) United States
(12) Patent Application Publication
(10) Pub. No.: US 2014/0005066 A1
(43) Pub. Date: Jan. 2, 2014

(54) Multiplexed PCR and Fluorescence Detection on a Droplet Actuator

(71) Applicant: ADVANCED LIQUID LOGIC INC., RESEARCH TRIANGLE PARK, NC (US)

(72) Inventors: Deborah Boles, Cary, NC (US); Lisa Perkins, Madison, WI (US); Allen E. Eckhardt, Durham, NC (US); Jennifer Foley, Cary, NC (US)

(73) Assignee: ADVANCED LIQUID LOGIC INC., Research Triangle Park, NC (US)

(21) Appl. No.: 13/932,719

(22) Filed: Jul. 1, 2013

Related U.S. Application Data
(60) Provisional application No. 61/666,490, filed on Jun. 29, 2012.

(51) Int. Cl. C12Q 1/70; C12Q 1/68

(52) U.S. Cl. CPC C12Q 1/703; C12Q 1/686; C12Q 1/701
USPC 506/9; 506/39

(57) ABSTRACT

The present invention provides a droplet actuator device and methods for multiplexed PCR amplification and detection of target amplicons within a single droplet. The methods of the invention combine quantitative real-time PCR (qPCR) amplification with fluorescence-based sequence specific detection technologies for amplified DNA. In one embodiment, fluorescently-labeled oligonucleotide probes may be used for hybridization-based multiplexed detection of target amplicons. The methods of the invention generally involve combining the necessary reactants to form a PCR-ready droplet and thermal cycling the droplet at temperatures sufficient to result in amplification of one or more target nucleic acids. Fluorescence-based detection techniques may be used for end-point or real-time analysis of DNA amplification. For end-point analysis, the accumulation of a signal, e.g., a fluorescence signal, is measured after the amplification of the target sequence is complete. For real-time analysis, the signal is measured while the amplification reaction is in progress.

### RELATED APPLICATIONS

This application is related and claims priority to U.S. Patent Application No. 61/666,490, filed on Jun. 29, 2012, entitled “Multiplexed PCR and Fluorescence Detection on a Droplet Actuator”, the entire disclosure of which is incorporated herein by reference.

### GOVERNMENT INTEREST

This invention was made with government support under HG004354 awarded by the National Institutes of Health of the United States. The government has certain rights in the invention.

### BACKGROUND

A droplet actuator typically includes one or more substrates configured to form a surface or gap for conducting droplet operations. The one or more substrates establish a droplet operations surface or gap for conducting droplet operations and may also include electrodes arrange to conduct the droplet operations. The droplet operations substrate or the gap between the substrates may be coated or filled with a filler fluid that is immiscible with the liquid that forms the droplets.

In one application, droplet actuators are used to conduct genetic analysis using polymerase chain reaction (PCR) technologies. In one example, PCR may be used for detection of one or more DNA target sequences in a sample. Analysis of the amplified target sequences (PCR amplicons) is often performed using sequence-dependent detection technologies, such as oligonucleotide probe hybridization. There is a need for techniques that make use of a droplet actuator for multiplexed amplification and detection of multiple target sequences in a single droplet.

### SUMMARY OF THE INVENTION

In one embodiment the invention provides a method for multiplexed real-time amplification and detection of multiple target sequences in a single droplet on a droplet actuator. The method may include loading a polymerase chain reaction (PCR) reaction mixture onto the droplet actuator; dispensing a first PCR reaction droplet onto a droplet operations surface of the droplet actuator having droplet operations electrodes arranged thereon; dispensing a second PCR reaction droplet onto the droplet operations surface of the droplet actuator, and combining the second PCR reaction droplet with the first PCR reaction droplet using droplet operations to form a combined PCR reaction droplet; thermal cycling the combined PCR reaction droplet to form an amplified sample droplet; and detecting fluorescence signals from the combined PCR reaction droplet during amplification. The first PCR reaction droplet may include a 1×PCR reaction droplet, the second PCR reaction droplet may include a 1×PCR reaction droplet, and the combined PCR reaction droplet may include a 2×PCR reaction droplet. The fluorescence signals from the amplified sample droplet may be detected in real time during amplification. The PCR reaction mixture may include sequence-specific DNA probes that are each labeled with a different fluorophore for detection of multiple target sequences in the single droplet. The PCR reaction mixture may include four (4) sequence-specific DNA probes each labeled with four (4) different fluorophores. The multiple target sequences may include an internal control sequence and one or more different target sequences. The amplified sample droplet following amplification may be transported using droplet operations to a recovery reservoir on the droplet actuator. The thermal cycling may include multiple cycles of the combined PCR reaction droplet through a plurality of temperature control zones, wherein the temperature control zones may include differing temperatures from one another. Loading the PCR reaction mixture onto the droplet actuator may include loading the PCR reaction mixture into a fluid dispensing reservoir of the droplet actuator. A cycle of the combined PCR reaction droplet thermal cycling, may include transporting the combined PCR reaction droplet using droplet operations to a first temperature control zone and incubating for a first period of time at a first temperature; transporting the combined PCR reaction droplet using droplet operations from the first temperature control zone to a second temperature control zone and incubating for a second period of time at a second temperature; and transporting the combined PCR reaction droplet using droplet operations from the second temperature control zone to a third temperature control zone and incubating for a third period of time at a third temperature. The combined PCR reaction droplet may include DNA. The first period of time is preferably sufficient for denaturation of DNA, the second period of time is preferably sufficient for primer and probe annealing, and the third period of time is preferably sufficient for primer elongation. The first period of time is preferably in the range of about seven (7) seconds, the second period of time is preferably in the range of about forty (40) seconds, and the third period of time is preferably in the range of about less than one (1) second. The first temperature is preferably sufficient for denaturation of DNA, the second temperature is preferably sufficient for primer and probe annealing, and the third temperature is preferably sufficient for primer elongation. The first temperature is preferably in the range of about 95° C., the second temperature is preferably in the range of about 55° C., and the third temperature is preferably in the range of about 72° C. The first period of time is preferably sufficient for denaturation of DNA may be determined using DNA melting analysis, including: labeling an amplified DNA sample with a fluorescent dye; loading the labeled amplified DNA sample onto the droplet actuator; dispensing and transporting the labeled amplified DNA sample using droplet operations to a detection spot within a temperature control zone on the droplet actuator heated to in the range of about 95° C.; measuring the fluorescence at regular time intervals until the fluorescence signal drops to a plateau level, wherein the time period to reach the plateau level correlates to an estimate of a required minimal PCR denaturation time. Detecting fluorescence signals may be conducted during the second period of time. Detecting fluorescence signals may include, transporting the combined PCR reaction droplet to a detection spot on the droplet actuator using droplet operations; activating a detection system; and measuring the fluorescence signals. The detection system may include a four channel detection system. Detecting fluorescence signals from the combined PCR reaction droplet preferably occurs in real-time during amplification. The detection spot may be smaller in size than the droplet operations electrode upon which the combined PCR reaction droplet is transported to. The may be used for multiplexed detection of one or more types of influenza virus and/or human immunodeficiency virus (HIV) in a whole blood sample.

In another embodiment, the invention provides a method for multiplexed amplification and detection of multiple target sequences in a single droplet on a droplet actuator. The method may include, loading an on-bench prepared PCR reaction mixture onto the droplet actuator; dispensing a droplet of the on-bench prepared PCR reaction mixture onto a droplet operations surface of the droplet actuator having droplet operations electrodes arranged thereon to form a sample droplet; thermal cycling the sample droplet to form an amplified sample droplet; and detecting fluorescence signals from the sample droplet during amplification.

In yet another embodiment, the invention provides a method of probe hybridization for detection of a target sequence in a single droplet on a droplet actuator. The method may include, amplifying a target sequence by PCR using a forward primer and a reverse primer; locating one or more probe sites between the forward primer and the reverse primer; providing a labeled probe designed to bind to a specific target sequence within the one or more probe sites; and extending the forward primer by a polymerase enzyme and degrading the labeled probe to produce a detectable signal. The exonuclease activity degrades the labeled probe. The detectable signal generated is preferably proportional to a concentration of the target sequence. The labeled probe may include a fluorescently-labeled probe. The labeled probe may include an oligonucleotide probe that is labeled with a fluorescent reporter molecule and a quencher molecule. The labeled probe may include a FRET probe. The target sequence may include a specific DNA sequence. The FRET probe may include a fluorophore covalently attached to a 5′-end of the oligonucleotide probe and a quencher molecule at a 3′-end. The FRET probe may be degraded by exonuclease activity and the fluorophore is preferably separated from the quencher producing a fluorescent signal.

In still yet another embodiment, the invention provides a detection system for detecting multiple different signals in a single sample droplet at a single detection spot on a droplet actuator. The detection system may include, an arrangement of multiple excitation light-emitting diodes (LEDs); and an arrangement of multiple detectors. The detection system may further include, multiple excitation lenses aligned with the multiple excitation LEDs, and structured to focus light emitted from a corresponding LED onto a corresponding excitation filter; multiple excitation filters aligned with the multiple excitation lenses, and structured each to select a different certain wavelength of light emitted from its corresponding LED for excitation of a certain signal; multiple mirrors aligned with the multiple excitation filters, and structured to direct the filtered light to a corresponding second directing mirror that is positioned in proximity of a corresponding excitation focusing lens; and wherein each directing mirror and corresponding focusing lens together multiplex the different wavelengths of light into a single excitation beam. The detection system may further include, multiple detection lenses aligned with the multiple detectors; multiple detection filters aligned with the multiple detection lenses; multiple dichroic filters aligned with the multiple detection filters, and structured to select a certain wavelength of light corresponding to an emission wavelength of a certain signal; multiple focusing lens aligned with the multiple dichroic filters; and wherein, the multiple focusing lens and directing mirrors together multiplex different wavelengths of light emitted from one or more signals from a single sample droplet into a single detection beam. The detection system may include a single excitation beam and a single detection beam directed to the single detection spot on the droplet actuator. The detection system is preferably capable of detecting at least four (4) different signals. The multiple different signals may include fluorescent signals. The detection system may be enclosed in a housing. The housing may be attached to a base plate adapted to position the detection system in a microfluidics system having the droplet actuator.

In still yet another embodiment, the invention provides a droplet actuator for multiplexed real-time amplification and detection of multiple target sequences in a single droplet. The droplet actuator may include, a bottom substrate separated from a top substrate to form a droplet operations gap, wherein the droplet operations gap is filled with a filler fluid; one or more fluid reservoirs; an electrode arrangement disposed on the bottom and/or top substrate comprising at least one of a path, line, and array of droplet operations electrodes; and a plurality of temperature control zones, wherein the temperature control zones may include differing temperatures from one another. The one or more fluid reservoirs may include at least one sample reservoir and one or more reagent reservoirs. The at least one sample reservoir and the one or more reagent reservoirs each may include an input port for loading fluids therein. The droplet operations electrodes may include electrowetting electrodes. The one or more reagent reservoirs may have a reagent dispensing electrode arranged with respect to the at least one of a path, line, and array of droplet operations electrodes. Each of the at least one sample reservoirs may have a sample dispensing electrode, wherein the sample dispensing electrode is preferably segmented into an arrangement of multiple individually controlled electrodes arranged with respect to the at least one of a path, line, and array of droplet operations electrodes. The at least one sample reservoir is preferably designed and structured to perform droplet operations, such as droplet mixing and/or droplet dispensing operations. The droplet actuator may further include one or more magnets positioned in proximity to certain of the droplet operations electrodes. The one or more magnets may be embedded within a deck that holds the droplet actuator. The one or more magnets may be positioned in a manner that ensures spatial immobilization of magnetically responsive beads during droplet dispensing operations. The droplet actuator may further include at least one of a sample dispensing region associated with the at least one sample reservoir and a droplet operations region associated with the one or more reagent reservoirs. The height of the gap between the bottom substrate and the top substrate at each region may vary.

In still yet another embodiment, the invention provides a system for performing multiplexed real-time amplification and detection of multiple target sequences in a single droplet on a droplet actuator. The system may include a processor for executing code and a memory in communication with the processor, the system comprising code stored in the memory that causes the processor at least to: load a polymerase chain reaction (PCR) reaction mixture onto the droplet actuator; dispense a first PCR reaction droplet onto a droplet operations surface of the droplet actuator having droplet operations electrodes arranged thereon; dispense a second PCR reaction droplet onto the droplet operations surface of the droplet actuator having droplet operations electrodes arranged thereon, and combining the second PCR reaction droplet with the first PCR reaction droplet using droplet operations to form a combined PCR reaction droplet; thermal cycle the combined PCR reaction droplet to form an amplified sample droplet; and detect fluorescence signals from the combined PCR reaction droplet during amplification.

In still yet another embodiment, the invention provides a computer readable medium storing processor executable instructions for performing a method of performing multiplexed real-time amplification and detection of multiple target sequences in a single droplet on a droplet actuator. The computer readable medium may include executable instructions including, loading a polymerase chain reaction (PCR) reaction mixture onto the droplet actuator; dispensing a first PCR reaction droplet onto a droplet operations surface of the droplet actuator having droplet operations electrodes arranged thereon; dispensing a second PCR reaction droplet onto the droplet operations surface of the droplet actuator having droplet operations electrodes arranged thereon, and combining the second PCR reaction droplet with the first PCR reaction droplet using droplet operations to form a combined PCR reaction droplet; thermal cycling the combined PCR reaction droplet to form an amplified sample droplet; and detecting fluorescence signals from the combined PCR reaction droplet during amplification.

In still yet another embodiment, the invention provides a method for integrated sample preparation and multiplexed detection of infectious agents on a droplet actuator. The method may include, combining a whole blood sample with a volume of a lysis buffer solution in a sample reservoir of the droplet actuator; incubating the combined whole blood sample and the volume of the lysis buffer solution for a period of time sufficient to yield a lysate comprising released nucleic acids; adding magnetically responsive nucleic acid capture beads suspended in a binding buffer to the lysate; incubating and mixing the magnetically responsive nucleic acid capture beads in the lysate for a period of time sufficient to allow binding of nucleic acids in the lysate to the magnetically responsive nucleic acid capture beads; washing the magnetically responsive nucleic acid capture beads to remove unbound material to yield a washed bead-containing droplet substantially lacking unbound material; repeating the washing to produce a concentrated sample droplet comprising purified RNA bound to the magnetically responsive nucleic acid capture beads; and transporting the concentrated sample droplet away from one or more magnets on the droplet actuator for further processing on the droplet actuator, wherein the further processing may include a method for multiplexed detection of infectious agents in a single droplet on the droplet actuator. The washing step may be repeated about 10 times. The method for multiplexed detection of infectious agents in a single droplet on the droplet actuator may include execution of a droplet based RT-qPCR PCR amplification protocol. The multiplexed detection of infectious agents may include detection of one or more types of influenza virus and/or human immunodeficiency virus (HIV).

In still yet another embodiment, the invention provides a method for integrated sample preparation and multiplexed detection of infectious agents on a droplet actuator. The method may include, combining a whole blood sample with a volume of a lysis buffer solution in a sample reservoir of the droplet actuator; incubating the combined whole blood sample and the volume of the lysis buffer solution for a period of time sufficient to yield a lysate comprising released nucleic acids; adding magnetically responsive nucleic acid capture beads suspended in a binding buffer to the lysate; incubating and mixing the magnetically responsive nucleic acid capture beads in the lysate for a period of time sufficient to allow binding of nucleic acids in the lysate to the magnetically responsive nucleic acid capture beads; washing the magnetically responsive nucleic acid capture beads to remove unbound material to yield a washed bead-containing droplet substantially lacking unbound material; repeating the washing to produce a concentrated sample droplet comprising purified RNA; eluting the purified RNA from the concentrated sample droplet with an elution buffer to produce eluted purified RNA; re-binding the eluted purified RNA to the magnetically responsive nucleic acid capture beads; repeating the washing eluting and re-binding steps to produce a droplet comprising eluted purified RNA; and transporting the droplet comprising eluted purified RNA away from the magnetically responsive nucleic acid capture beads for further processing on the droplet actuator, wherein the further processing may include a method for multiplexed detection of infectious agents in a single droplet on the droplet actuator. The washing step may be repeated about 10 times. The method for multiplexed detection of infectious agents in a single droplet on the droplet actuator may include execution of a droplet based RT-qPCR PCR amplification protocol. The multiplexed detection of infectious agents may include detection of one or more types of influenza virus and/or human immunodeficiency virus (HIV).

In still yet another embodiment, the invention provides a method for integrated sample preparation and multiplexed detection of infectious agents on a droplet actuator. The method may include, combining a whole blood sample with a volume of a lysis buffer solution and a volume of an agglutinate in a sample reservoir of the droplet actuator; incubating the combined whole blood sample, the volume of the lysis buffer solution, and the agglutinate for a period of time sufficient for agglutination and to yield a lysate comprising released nucleic acids; adding magnetically responsive nucleic acid capture beads suspended in a binding buffer to the lysate; incubating and mixing the magnetically responsive nucleic acid capture beads in the lysate for a period of time sufficient to allow binding of nucleic acids in the lysate to the magnetically responsive nucleic acid capture beads; concentrating the magnetically responsive nucleic acid capture beads and dispensing a concentrated sample droplet comprising the magnetically responsive nucleic acid capture beads onto a droplet operations electrode, wherein the droplet operations electrode is between flanking electrodes and near at least one magnet; washing the magnetically responsive nucleic acid capture beads to remove unbound material to yield a washed bead-containing droplet substantially lacking unbound material; repeating the washing step to produce a concentrated sample droplet comprising purified RNA bound to the magnetically responsive nucleic acid capture beads; transporting the concentrated sample droplet away from one or more magnets on the droplet actuator for further processing on the droplet actuator, wherein the further processing may include a method for multiplexed detection of infectious agents in a single droplet on the droplet actuator. The agglutinate may be erythroagglutinin PHA-E. Prior to step the washing step, the droplet operations electrode may be flooded with wash buffer to facilitate dilution and microfluidic operations. The washing step may be repeated from about 10 times to about 30 times. The method for multiplexed detection of infectious agents in a single droplet on the droplet actuator may include execution of a droplet based RT-qPCR PCR amplification protocol. The multiplexed detection of infectious agents may include detection of one or more types of influenza virus and/or human immunodeficiency virus (HIV).

In still yet another embodiment, the invention provides a method for integrated sample preparation and multiplexed detection of infectious agents on a droplet actuator. The method may include, combining a whole blood sample with a volume of a lysis buffer solution and a volume of an agglutinate in a sample reservoir of the droplet actuator; incubating the combined whole blood sample, the volume of the lysis buffer solution, and the agglutinate for a period of time sufficient for agglutination and to yield a lysate comprising released nucleic acids; adding magnetically responsive nucleic acid capture beads suspended in a binding buffer to the lysate; incubating and mixing the magnetically responsive nucleic acid capture beads in the lysate for a period of time sufficient to allow binding of nucleic acids in the lysate to the magnetically responsive nucleic acid capture beads; concentrating the magnetically responsive nucleic acid capture beads and dispensing a concentrated sample droplet comprising the magnetically responsive nucleic acid capture beads onto a droplet operations electrode, wherein the droplet operations electrode is between flanking electrodes and near at least one magnet; washing the magnetically responsive nucleic acid capture beads in the concentrated sample droplet to remove unbound material to yield a washed bead-containing droplet substantially lacking unbound material; repeating the washing step to produce a concentrated sample droplet comprising purified RNA; eluting the purified RNA from the concentrated sample droplet with an elution buffer to produce eluted purified RNA; and transporting the eluted purified RNA away from the magnetically responsive nucleic acid capture beads for further processing on the droplet actuator, wherein the further processing may include a method for multiplexed detection of infectious agents in a single droplet on the droplet actuator. The agglutinate may be erythroagglutinin PHA-E. Prior to the washing step, the droplet operations electrode may be flooded with wash buffer to facilitate dilution and microfluidic operations. The washing step may be repeated from about 10 times to about 30 times. The method for multiplexed detection of infectious agents in a single droplet on the droplet actuator may include execution of a droplet based RT-qPCR PCR amplification protocol. The multiplexed detection of infectious agents comprises detection of one or more types of influenza virus and/or human immunodeficiency virus (HIV).

### DEFINITIONS

As used herein, the following terms have the meanings indicated.

“Activate,” with reference to one or more electrodes, means affecting a change in the electrical state of the one or more electrodes which, in the presence of a droplet, results in a droplet operation. Activation of an electrode can be accomplished using alternating or direct current. Any suitable voltage may be used. For example, an electrode may be activated using a voltage which is greater than about 150 V, or greater than about 200 V, or greater than about 250 V, or from about 275 V to about 375 V, or about 300 V. Where alternating current is used, any suitable frequency may be employed. For example, an electrode may be activated using alternating current having a frequency from about 1 Hz to about 100 Hz, or from about 10 Hz to about 60 Hz, or from about 20 Hz to about 40 Hz, or about 30 Hz.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical, amorphous and other three dimensional shapes. The bead may, for example, be capable of being subjected to a droplet operation in a droplet on a droplet actuator or otherwise configured with respect to a droplet actuator in a manner which permits a droplet on the droplet actuator to be brought into contact with the bead on the droplet actuator and/or off the droplet actuator. Beads may be provided in a droplet, in a droplet operations gap, or on a droplet operations surface. Beads may be provided in a reservoir that is external to a droplet operations gap or situated apart from a droplet operations surface, and the reservoir may be associated with a fluid path that permits a droplet including the beads to be brought into a droplet operations gap or into contact with a droplet operations surface. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead, a portion of a bead, or only one component of a bead. The remainder of the bead may include, among other things, polymeric material, coatings, and moieties which permit attachment of an assay reagent. Examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Patent Publication Nos. 20050260686, entitled “Multiplex flow assays preferably with magnetic particles as solid phase,” published on Nov. 24, 2005; 20030132538, entitled “Encapsulation of discrete quanta of fluorescent particles,” published on Jul. 17, 2003; 20050118574, entitled “Multiplexed Analysis of Clinical Specimens Apparatus and Method,” published on Jun. 2, 2005; 20050277197. Entitled “Microparticles with Multiple Fluorescent Signals and Methods of Using Same,” published on Dec. 15, 2005; 20060159962, entitled “Magnetic Microspheres for use in Fluorescence-based Applications,” published on Jul. 20, 2006; the entire disclosures of which are incorporated herein by reference for their teaching concerning beads and magnetically responsive materials and beads. Beads may be pre-coupled with a biomolecule or other substance that is able to bind to and form a complex with a biomolecule. Beads may be pre-coupled with an antibody, protein or antigen, DNA/RNA probe or any other molecule with an affinity for a desired target. Examples of droplet actuator techniques for immobilizing magnetically responsive beads and/or non-magnetically responsive beads and/or conducting droplet operations protocols using beads are described in U.S. patent application Ser. No. 11/639,566, entitled “Droplet-Based Particle Sorting,” filed on Dec. 15, 2006; U.S. Patent Application No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; International Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference. Bead characteristics may be employed in the multiplexing aspects of the invention. Examples of beads having characteristics suitable for multiplexing, as well as methods of detecting and analyzing signals emitted from such beads, may be found in U.S. Patent Publication No. 20080305481, entitled “Systems and Methods for Multiplex Analysis of PCR in Real Time,” published on Dec. 11, 2008; U.S. Patent Publication No. 20080151240, “Methods and Systems for Dynamic Range Expansion,” published on Jun. 26, 2008; U.S. Patent Publication No. 20070207513, entitled “Methods, Products, and Kits for Identifying an Analyte in a Sample,” published on Sep. 6, 2007; U.S. Patent Publication No. 20070064990, entitled “Methods and Systems for Image Data Processing,” published on Mar. 22, 2007; U.S. Patent Publication No. 20060159962, entitled “Magnetic Microspheres for use in Fluorescence-based Applications,” published on Jul. 20, 2006; U.S. Patent Publication No. 20050277197, entitled “Microparticles with Multiple Fluorescent Signals and Methods of Using Same,” published on Dec. 15, 2005; and U.S. Patent Publication No. 20050118574, entitled “Multiplexed Analysis of Clinical Specimens Apparatus and Method,” published on Jun. 2, 2005.

“Droplet” means a volume of liquid on a droplet actuator. Typically, a droplet is at least partially bounded by a filler fluid. For example, a droplet may be completely surrounded by a filler fluid or may be bounded by filler fluid and one or more surfaces of the droplet actuator. As another example, a droplet may be bounded by filler fluid, one or more surfaces of the droplet actuator, and/or the atmosphere. As yet another example, a droplet may be bounded by filler fluid and the atmosphere. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, combinations of such shapes, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids.

“Droplet Actuator” means a device for manipulating droplets. For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on filed on Jan. 30, 2006; Pollack et al., International Patent Application No. PCT/US2006/047486, entitled “Droplet-Based Biochemistry,” filed on Dec. 11, 2006; Shenderov, U.S. Pat. Nos. 6,773,566, entitled “Electrostatic Actuators for Microfluidics and Methods for Using Same,” issued on Aug. 10, 2004 and 6,565,727, entitled “Actuators for Microfluidics Without Moving Parts,” issued on Jan. 24, 2000; Kim and/or Shah et al., U.S. patent application Ser. Nos. 10/343,261, entitled “Electrowetting-driven Micropumping,” filed on Jan. 27, 2003, 11/275,668, entitled “Method and Apparatus for Promoting the Complete Transfer of Liquid Drops from a Nozzle,” filed on Jan. 23, 2006, 11/460,188, entitled “Small Object Moving on Printed Circuit Board,” filed on Jan. 23, 2006, 12/465,935, entitled “Method for Using Magnetic Particles in Droplet Microfluidics,” filed on May 14, 2009, and 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Microfluidic Control via Electrowetting, for Chemical, Biochemical and Biological Assays and the Like,” issued on Jan. 16, 2007; Becker and Gascoyne et al., U.S. Pat. Nos. 7,641,779, entitled “Method and Apparatus for Programmable fluidic Processing,” issued on Jan. 5, 2010, and 6,977,033, entitled “Method and Apparatus for Programmable fluidic Processing,” issued on Dec. 20, 2005; Decre et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, International Patent Pub. No. WO/2009/003184, entitled “Digital Microfluidics Based Apparatus for Heat-exchanging Chemical Processes,” published on Dec. 31, 2008; Fouillet et al., U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels Electronic Liquid Transport with Continuous Channel Functionality,” Lab Chip, 10:832-836 (2010); the entire disclosures of which are incorporated herein by reference, along with their priority documents. Certain droplet actuators will include one or more substrates arranged with a gap therebetween and electrodes associated with (e.g., layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, dielectric layers and/or the electrodes forming a droplet operations surface. A top substrate may also be provided, which is separated from the droplet operations surface by a gap, commonly referred to as a droplet operations gap. Various electrode arrangements on the top and/or bottom substrates are discussed in the above-referenced patents and applications and certain novel electrode arrangements are discussed in the description of the invention. During droplet operations it is preferred that droplets remain in continuous contact or frequent contact with a ground or reference electrode. A ground or reference electrode may be associated with the top substrate facing the gap, the bottom substrate facing the gap, in the gap. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator. In one embodiment, a conductive material (e.g., an epoxy, such as MASTER BOND™ Polymer System EP79, available from Master Bond, Inc., Hackensack, N.J.) provides the electrical connection between electrodes on one substrate and electrical paths on the other substrates, e.g., a ground electrode on a top substrate may be coupled to an electrical path on a bottom substrate by such a conductive material. Where multiple substrates are used, a spacer may be provided between the substrates to determine the height of the gap therebetween and define dispensing reservoirs. The spacer height may, for example, be from about 5 μm to about 600 μm, or about 100 μm to about 400 μm, or about 200 μm to about 350 μm, or about 250 μm to about 300 μm, or about 275 μm. The spacer may, for example, be formed of a layer of projections form the top or bottom substrates, and/or a material inserted between the top and bottom substrates. One or more openings may be provided in the one or more substrates for forming a fluid path through which liquid may be delivered into the droplet operations gap. The one or more openings may in some cases be aligned for interaction with one or more electrodes, e.g., aligned such that liquid flowed through the opening will come into sufficient proximity with one or more droplet operations electrodes to permit a droplet operation to be effected by the droplet operations electrodes using the liquid. The base (or bottom) and top substrates may in some cases be formed as one integral component. One or more reference electrodes may be provided on the base (or bottom) and/or top substrates and/or in the gap. Examples of reference electrode arrangements are provided in the above referenced patents and patent applications. In various embodiments, the manipulation of droplets by a droplet actuator may be electrode mediated, e.g., electrowetting mediated or dielectrophoresis mediated or Coulombic force mediated. Examples of other techniques for controlling droplet operations that may be used in the droplet actuators of the invention include using devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed to conduct a droplet operation in a droplet actuator of the invention. Similarly, one or more of the foregoing may be used to deliver liquid into a droplet operations gap, e.g., from a reservoir in another device or from an external reservoir of the droplet actuator (e.g., a reservoir associated with a droplet actuator substrate and a fluid path from the reservoir into the droplet operations gap). Droplet operations surfaces of certain droplet actuators of the invention may be made from hydrophobic materials or may be coated or treated to make them hydrophobic. For example, in some cases some portion or all of the droplet operations surfaces may be derivatized with low surface-energy materials or chemistries, e.g., by deposition or using in situ synthesis using compounds such as poly- or per-fluorinated compounds in solution or polymerizable monomers. Examples include TEFLON® AF (available from DuPont, Wilmington, Del.), members of the cytop family of materials, coatings in the FLUOROPEL® family of hydrophobic and superhydrophobic coatings (available from Cytonix Corporation, Beltsville, Md.), silane coatings, fluorosilane coatings, hydrophobic phosphonate derivatives (e.g., those sold by Aculon, Inc), and NOVEC™ electronic coatings (available from 3M Company, St. Paul, Minn.), and other fluorinated monomers for plasma-enhanced chemical vapor deposition (PECVD). In some cases, the droplet operations surface may include a hydrophobic coating having a thickness ranging from about 10 nm to about 1,000 nm. Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer that is deposited onto a plastic substrate may be poly(3,4-ethylenedioxythiophene) poly(styrenesulfonate) (PEDOT:PSS). Other examples of electrically conducting organic polymers and alternative conductive layers are described in Pollack et al., International Patent Application No. PCT/US2010/040705, entitled “Droplet Actuator Devices and Methods,” the entire disclosure of which is incorporated herein by reference. One or both substrates may be fabricated using a printed circuit board (PCB), glass, indium tin oxide (ITO)-coated glass, and/or semiconductor materials as the substrate. When the substrate is ITO-coated glass, the ITO coating is preferably a thickness in the range of about 20 to about 200 nm, preferably about 50 to about 150 nm, or about 75 to about 125 nm, or about 100 nm. In some cases, the top and/or bottom substrate includes a PCB substrate that is coated with a dielectric, such as a polyimide dielectric, which may in some cases also be coated or otherwise treated to make the droplet operations surface hydrophobic. When the substrate includes a PCB, the following materials are examples of suitable materials: MITSUI™ BN-300 (available from MITSUI Chemicals America, Inc., San Jose Calif.); ARLON™ 11N (available from Arlon, Inc, Santa Ana, Calif.).; NELCO® N4000-6 and N5000-30/32 (available from Park Electrochemical Corp., Melville, N.Y.); ISOLA™ FR406 (available from Isola Group, Chandler, Ariz.), especially IS620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT® nonwoven aramid reinforcement (available from DuPont, Wilmington, Del.); NOMEX® brand fiber (available from DuPont, Wilmington, Del.); and paper. Various materials are also suitable for use as the dielectric component of the substrate. Examples include: vapor deposited dielectric, such as PARYLENE™ C (especially on glass) and PARYLENE™ N (available from Parylene Coating Services, Inc., Katy, Tex.); TEFLON® AF coatings; cytop; soldermasks, such as liquid photoimageable soldermasks (e.g., on PCB) like TAIYO™ PSR4000 series, TAIYO™ PSR and AUS series (available from Taiyo America, Inc. Carson City, Nev.) (good thermal characteristics for applications involving thermal control), and PROBIMER™ 8165 (good thermal characteristics for applications involving thermal control (available from Huntsman Advanced Materials Americas Inc., Los Angeles, Calif.); dry film soldermask, such as those in the VACREL® dry film soldermask line (available from DuPont, Wilmington, Del.); film dielectrics, such as polyimide film (e.g., KAPTON® polyimide film, available from DuPont, Wilmington, Del.), polyethylene, and fluoropolymers (e.g., FEP), polytetrafluoroethylene; polyester; polyethylene naphthalate; cyclo-olefin copolymer (COC); cyclo-olefin polymer (COP); any other PCB substrate material listed above; black matrix resin; and polypropylene. Droplet transport voltage and frequency may be selected for performance with reagents used in specific assay protocols. Design parameters may be varied, e.g., number and placement of on-actuator reservoirs, number of independent electrode connections, size (volume) of different reservoirs, placement of magnets/bead washing zones, electrode size, inter-electrode pitch, and gap height (between top and bottom substrates) may be varied for use with specific reagents, protocols, droplet volumes, etc. In some cases, a substrate of the invention may derivatized with low surface-energy materials or chemistries, e.g., using deposition or in situ synthesis using poly- or per-fluorinated compounds in solution or polymerizable monomers. Examples include TEFLON® AF coatings and FLUOROPEL® coatings for dip or spray coating, and other fluorinated monomers for plasma-enhanced chemical vapor deposition (PECVD). Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray industries, Inc., Japan. Electrodes of a droplet actuator are typically controlled by a controller or a processor, which is itself provided as part of a system, which may include processing functions as well as data and software storage and input and output capabilities. Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution. An example of reconstitutable reagents suitable for use with the invention includes those described in Meathrel, et al., U.S. Pat. No. 7,727,466, entitled “Disintegratable films for diagnostic devices,” granted on Jun. 1, 2010.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.” Impedance or capacitance sensing or imaging techniques may sometimes be used to determine or confirm the outcome of a droplet operation. Examples of such techniques are described in Sturmer et al., International Patent Pub. No. WO/2008/101194, entitled “Capacitance Detection in a Droplet Actuator,” published on Aug. 21, 2008, the entire disclosure of which is incorporated herein by reference. Generally speaking, the sensing or imaging techniques may be used to confirm the presence or absence of a droplet at a specific electrode. For example, the presence of a dispensed droplet at the destination electrode following a droplet dispensing operation confirms that the droplet dispensing operation was effective. Similarly, the presence of a droplet at a detection spot at an appropriate step in an assay protocol may confirm that a previous set of droplet operations has successfully produced a droplet for detection. Droplet transport time can be quite fast. For example, in various embodiments, transport of a droplet from one electrode to the next may exceed about 1 sec, or about 0.1 sec, or about 0.01 sec, or about 0.001 sec. In one embodiment, the electrode is operated in AC mode but is switched to DC mode for imaging. It is helpful for conducting droplet operations for the footprint area of droplet to be similar to electrowetting area; in other words, 1×-, 2×- 3×-droplets are usefully controlled operated using 1, 2, and 3 electrodes, respectively. If the droplet footprint is greater than the number of electrodes available for conducting a droplet operation at a given time, the difference between the droplet size and the number of electrodes should typically not be greater than 1; in other words, a 2× droplet is usefully controlled using 1 electrode and a 3× droplet is usefully controlled using 2 electrodes. When droplets include beads, it is useful for droplet size to be equal to the number of electrodes controlling the droplet, e.g., transporting the droplet.

“Filler fluid” means a fluid associated with a droplet operations substrate of a droplet actuator, which fluid is sufficiently immiscible with a droplet phase to render the droplet phase subject to electrode-mediated droplet operations. For example, the gap of a droplet actuator is typically filled with a filler fluid. The filler fluid may, for example, be a low-viscosity oil, such as silicone oil or hexadecane filler fluid. The filler fluid may fill the entire gap of the droplet actuator or may coat one or more surfaces of the droplet actuator. Filler fluids may be conductive or non-conductive. Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents used in the specific assay protocols and effective interaction or non-interaction with droplet actuator materials. Examples of filler fluids and filler fluid formulations suitable for use with the invention are provided in Srinivasan et al, International Patent Pub. Nos. WO/2010/027894, entitled “Droplet Actuators, Modified Fluids and Methods,” published on Mar. 11, 2010, and WO/2009/021173, entitled “Use of Additives for Enhancing Droplet Operations,” published on Feb. 12, 2009; Sista et al., International Patent Pub. No. WO/2008/098236, entitled “Droplet Actuator Devices and Methods Employing Magnetic Beads,” published on Aug. 14, 2008; and Monroe et al., U.S. Patent Publication No. 20080283414, entitled “Electrowetting Devices,” filed on May 17, 2007; the entire disclosures of which are incorporated herein by reference, as well as the other patents and patent applications cited herein.

“Immobilize” with respect to magnetically responsive beads, means that the beads are substantially restrained in position in a droplet or in filler fluid on a droplet actuator. For example, in one embodiment, immobilized beads are sufficiently restrained in position in a droplet to permit execution of a droplet splitting operation, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe₁₂O₁₉, CoO, NiO, Mn₂O₃, Cr₂O₃, and CoMnP.

“Reservoir” means an enclosure or partial enclosure configured for holding, storing, or supplying liquid. A droplet actuator system of the invention may include on-cartridge reservoirs and/or off-cartridge reservoirs. On-cartridge reservoirs may be (1) on-actuator reservoirs, which are reservoirs in the droplet operations gap or on the droplet operations surface; (2) off-actuator reservoirs, which are reservoirs on the droplet actuator cartridge, but outside the droplet operations gap, and not in contact with the droplet operations surface; or (3) hybrid reservoirs which have on-actuator regions and off-actuator regions. An example of an off-actuator reservoir is a reservoir in the top substrate. An off-actuator reservoir is typically in fluid communication with an opening or fluid path arranged for flowing liquid from the off-actuator reservoir into the droplet operations gap, such as into an on-actuator reservoir. An off-cartridge reservoir may be a reservoir that is not part of the droplet actuator cartridge at all, but which flows liquid to some portion of the droplet actuator cartridge. For example, an off-cartridge reservoir may be part of a system or docking station to which the droplet actuator cartridge is coupled during operation. Similarly, an off-cartridge reservoir may be a reagent storage container or syringe which is used to force fluid into an on-cartridge reservoir or into a droplet operations gap. A system using an off-cartridge reservoir will typically include a fluid passage means whereby liquid may be transferred from the off-cartridge reservoir into an on-cartridge reservoir or into a droplet operations gap.

“Transporting into the magnetic field of a magnet,” “transporting towards a magnet,” and the like, as used herein to refer to droplets and/or magnetically responsive beads within droplets, is intended to refer to transporting into a region of a magnetic field capable of substantially attracting magnetically responsive beads in the droplet. Similarly, “transporting away from a magnet or magnetic field,” “transporting out of the magnetic field of a magnet,” and the like, as used herein to refer to droplets and/or magnetically responsive beads within droplets, is intended to refer to transporting away from a region of a magnetic field capable of substantially attracting magnetically responsive beads in the droplet, whether or not the droplet or magnetically responsive beads is completely removed from the magnetic field. It will be appreciated that in any of such cases described herein, the droplet may be transported towards or away from the desired region of the magnetic field, and/or the desired region of the magnetic field may be moved towards or away from the droplet. Reference to an electrode, a droplet, or magnetically responsive beads being “within” or “in” a magnetic field, or the like, is intended to describe a situation in which the electrode is situated in a manner which permits the electrode to transport a droplet into and/or away from a desired region of a magnetic field, or the droplet or magnetically responsive beads is/are situated in a desired region of the magnetic field, in each case where the magnetic field in the desired region is capable of substantially attracting any magnetically responsive beads in the droplet. Similarly, reference to an electrode, a droplet, or magnetically responsive beads being “outside of” or “away from” a magnetic field, and the like, is intended to describe a situation in which the electrode is situated in a manner which permits the electrode to transport a droplet away from a certain region of a magnetic field, or the droplet or magnetically responsive beads is/are situated away from a certain region of the magnetic field, in each case where the magnetic field in such region is not capable of substantially attracting any magnetically responsive beads in the droplet or in which any remaining attraction does not eliminate the effectiveness of droplet operations conducted in the region. In various aspects of the invention, a system, a droplet actuator, or another component of a system may include a magnet, such as one or more permanent magnets (e.g., a single cylindrical or bar magnet or an array of such magnets, such as a Halbach array) or an electromagnet or array of electromagnets, to form a magnetic field for interacting with magnetically responsive beads or other components on chip. Such interactions may, for example, include substantially immobilizing or restraining movement or flow of magnetically responsive beads during storage or in a droplet during a droplet operation or pulling magnetically responsive beads out of a droplet.

“Washing” with respect to washing a bead means reducing the amount and/or concentration of one or more substances in contact with the bead or exposed to the bead from a droplet in contact with the bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### BRIEF DESCRIPTION OF THE DRAWINGS

FIG. 1 illustrates a flow diagram of an example of a microfluidic protocol for multiplexed PCR in a single droplet on a droplet actuator;

FIG. 2 illustrates a flow diagram of an example of a thermal cycling protocol for multiplexed PCR on a droplet actuator;

FIG. 3 illustrates a flow diagram of an example of a detection protocol for detection of multiple (e.g., four) fluorophores in a single reaction droplet;

FIGS. 4A and 4B illustrate an example of a process of amplification and probe hybridization for detection of a target sequence;

FIG. 5 illustrates an example of a primer/probe arrangement for multiplexed PCR amplification of the 109 bp fragment of the E. coli polymerase III subunit α gene;

FIG. 6 shows an example of a plot of fluorescence data from eight separate PCR amplifications each using a single FRET detection probe;

FIG. 7 shows an example of a plot of fluorescence data of a comparison of fluorophore/quencher pair on probe properties during PCR amplification;

FIG. 8 shows examples of multiple plots (i.e., plots A through F) of fluorescence data from qPCR amplifications comparing single probe and double probe (two-plex) reactions;

FIGS. 9, 10 and 11 illustrate various views of an example a detection system for detection of multiple (e.g., four) different fluorophores in a sample droplet at a single detection spot.

FIGS. 12A and 12B show examples of bar graphs of FAM and HEX fluorophore calibration data, respectively, obtained on-actuator;

FIGS. 13A and 13B show examples of bar graphs of TR and Cy5 fluorophore calibration data, respectively, obtained on-actuator;

FIG. 14 shows a screenshot of an example of an overlay of a multiplexed fluorophore emission and detection spectra;

FIGS. 15A and 15B show examples of plots of fluorescence data from multiplexed RT-qPCR amplification of influenza A, influenza B, and MS2 (internal process control) generated on-actuator and on-bench, respectively;

FIG. 16 shows an example of a plot of fluorescence data from multiplexed RT-qPCR amplification of influenza A and MS2 (internal control) in a human sample matrix;

FIGS. 17A and 17B illustrate top views of a portion of an example of a droplet actuator and show a process of dispensing and concentrating magnetically responsive capture beads in a droplet of whole blood lysate and plasma-like (agglutinated) lysate, respectively; and

FIG. 18 shows an example of a plot of fluorescence data from HIV RNA isolated from a whole blood sample and amplified on a droplet actuator.

### DESCRIPTION

The present invention provides a droplet actuator device and methods for multiplexed PCR amplification and detection of target amplicons within a single droplet. For example, the methods of the invention combine quantitative real-time PCR (qPCR) amplification with fluorescence-based sequence specific detection technologies for amplified DNA. In one embodiment, fluorescently-labeled oligonucleotide probes may be used for hybridization-based multiplexed detection of target amplicons. The methods of the invention generally involve combining the necessary reactants to form a PCR-ready droplet and thermal cycling the droplet at temperatures sufficient to result in amplification of one or more target nucleic acids. Fluorescence-based detection techniques may be used for end-point or real-time analysis of DNA amplification. For end-point analysis, the accumulation of a signal, e.g., a fluorescence signal, is measured after the amplification of the target sequence is complete. For real-time analysis, the signal is measured while the amplification reaction is in progress.

In another embodiment, the invention provides a detection system that uses a single excitation beam and a single detection beam to collect multiple (e.g., four) different fluorescent signals from a droplet at a single detection spot on a droplet actuator.

In yet another embodiment, the invention provides a droplet actuator device and methods for integrated sample preparation and multiplexed detection of an infectious agent, such as influenza virus and HIV in a biological sample (e.g., a whole blood sample).

7.1 Multiplexed qPCR on a Droplet Actuator

Because of the flexibility and programmability of a digital microfluidics platform, multiplexed quantitative real-time PCR (qPCR) assays may be readily performed. Rapid PCR thermocycling is performed in flow-through format where for each cycle the reaction droplets are cyclically transported between different temperature zones within the oil filled droplet actuator. In one embodiment, sequence-specific DNA probes that are labeled with different fluorophores may be used during PCR amplification for detection of multiple target sequences (e.g., 4 different target sequences) in a single droplet. In one example, the multiple target sequences in a single droplet may include an internal DNA control sequence and three different target sequences of interest. Because multiple target sequences are multiplexed and analyzed in a single droplet, multiple sample droplets (e.g., the same sample or multiple different samples) may be run in parallel on a droplet actuator.

7.1.1 Digital Microfluidics qPCR Protocol

Digital microfluidic technology conducts droplet operations on discrete droplets by electrical control of their surface tension (electrowetting). The droplets may be sandwiched between two substrates, a bottom substrate and a top substrate separated by a gap. The bottom substrate may, for example, be a printed circuit board (PCB) with an arrangement of electrically addressable electrodes. The top substrate may, for example, be an injection molded plastic top substrate that has a reference electrode plane made, for example, from conductive ink or indium tin oxide (ITO). The bottom substrate and the top substrate may be coated with a hydrophobic material. The space around the droplets (i.e., the gap between bottom and top substrates) may be filled with an immiscible inert fluid, such as silicone oil (e.g., 7 cST with 0.005% Span 85), to prevent evaporation of the droplets and to facilitate their transport within the device. An electric field, formed when voltage is applied to a droplet operations electrode on the bottom substrate, reduces the interfacial tension between the droplet and the electrode. This effect may be used to transport droplets using surface energy gradients established by activating a pattern of droplet operations electrodes on the bottom substrate along any path of contiguous electrodes. Other droplet operations may be effected by varying the patterns of voltage activation; examples include merging, splitting, mixing, and dispensing of droplets.

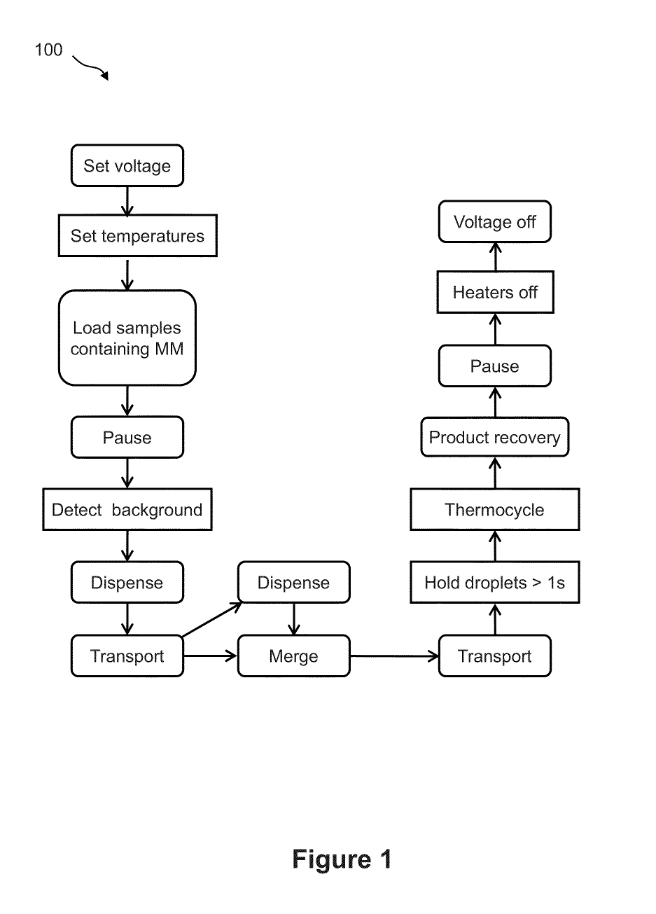

FIG. 1 illustrates a flow diagram of an example of a microfluidic protocol 100 for multiplexed PCR in a single droplet on a droplet actuator. In this example, a PCR reaction is prepared on bench by combining a PCR master mix (e.g., primer sets, dNTPs, enzyme, fluorescent probes, Taq polymerase and buffer) with an aliquot of a DNA sample. The PCR reaction mixture is then loaded into a fluid dispensing reservoir of a droplet actuator.

Microfluidic protocol 100 for multiplexed PCR in a single droplet may include, but is not limited to, the following steps.

In certain steps, electrowetting voltage and reaction zone temperatures are set.

In other steps, the PCR reaction(s) prepared on-bench is loaded into a fluid dispensing reservoir of a droplet actuator. Background fluorescence is determined.

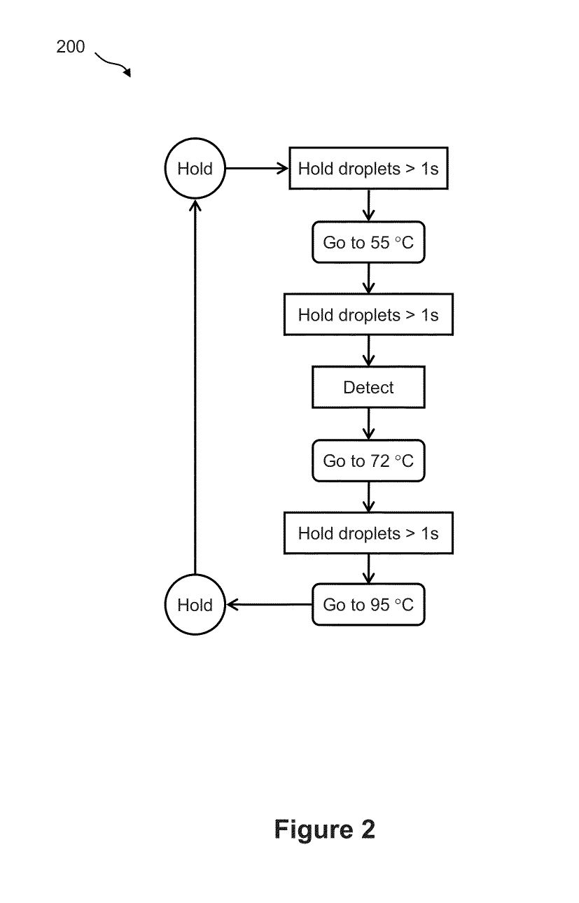

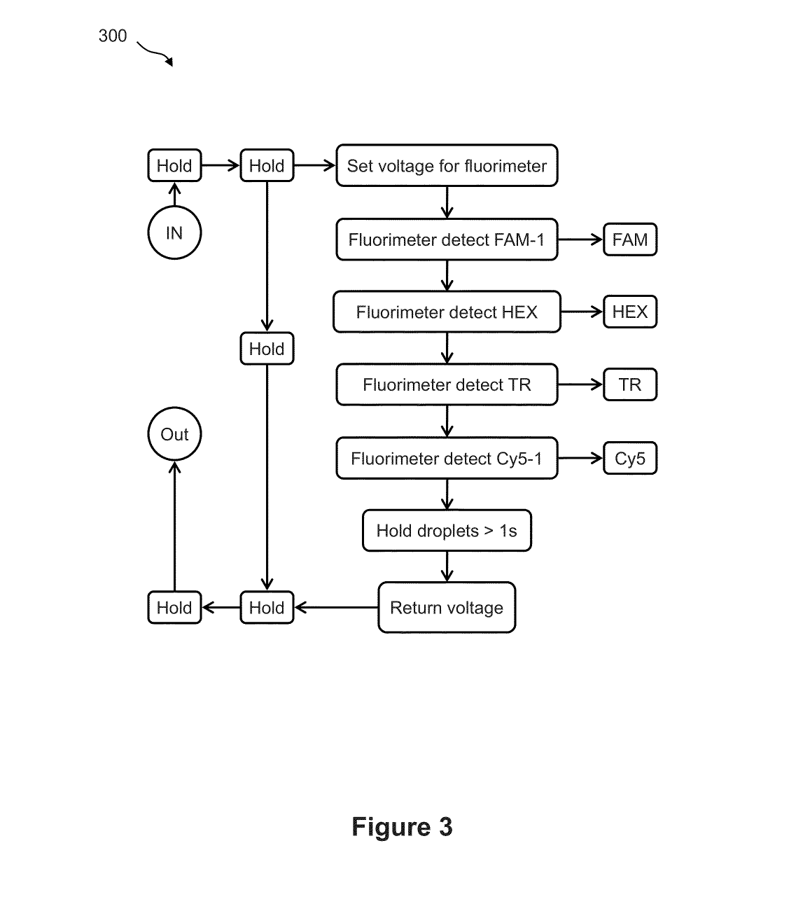

In other steps, a first 1×PCR reaction droplet is dispensed and transported using droplet operations to a certain droplet operations electrode. A second 1×PCR reaction droplet is dispensed and combined (merged) using droplet operations with the first 1×PCR reaction droplet to yield a 2× reaction droplet. The 2× reaction droplet is transported using droplet operations to a thermal cycling zone on the droplet actuator. The thermal cycling zone may include three temperature control zones. One temperature control zone may be heated to about 95° C., which is a temperature sufficient for denaturation of double stranded DNA. A second temperature control zone may be heated to about 55° C., which is a temperature sufficient for primer and probe annealing. A third temperature control zone may be heated to about 72° C., which is a temperature sufficient for primer elongation. To initiate the PCR amplification reaction, the 2× reaction droplet is transported using droplet operations to the 95° C. temperature control zone. After an incubation period (e.g., 2 minutes), amplification is performed by thermal cycling the 2× reaction droplet, as described in reference to FIG. 2. The fluorescence signal is detected in real time during amplification, as described in reference to FIG. 3.

In other steps, after completion of the amplification reaction, the 2× reaction droplet is transported using droplet operations to a product recovery reservoir on the droplet actuator. Heaters and electrowetting voltage are turned off.

FIG. 2 illustrates a flow diagram of an example of a thermal cycling protocol 200 for multiplexed PCR on a droplet actuator. In one example, thermal cycling protocol 200 may include 45 cycles in which each cycle may include 7 seconds at 95° C., 40 seconds at 55° C., and less than 1 second at 72° C. Thermal cycling protocol 200 may include, but is not limited to, the following steps.

In one step, the 2× reaction droplet is transported using droplet operations from the 95° C. temperature control zone to the 55° C. temperature control zone. The 2× reaction droplet is incubated at 55° C. for 40 seconds. The fluorescence signal is detected as described in reference to FIG. 3.

In another step, the 2× reaction droplet is transported using droplet operations from the 55° C. temperature control zone to the 72° C. temperature control zone. The 2× reaction droplet is incubated at 72° C. for less than 1 second.

In another step, the 2× reaction droplet is transported using droplet operations from the 72° C. temperature control zone to the 95° C. temperature control zone. The 2× reaction droplet is incubated at 95° C. for 7 seconds. The thermal cycling protocol is repeated (e.g., 45 cycles).

DNA denaturation time in thermal cycling protocol 200 may be selected to provide reduced total assay time and less bubble formation. DNA denaturation time may, for example, be determined using DNA melting analysis. One example of a protocol to determine DNA denaturation time may include, but is not limited to, the following: A DNA sample is amplified on-bench and labeled with a fluorescent DNA intercalating dye, such as EvaGreen (available from Biotium Inc, Hayward, Calif.). The amplified DNA sample is loaded into a fluid dispensing reservoir of a droplet actuator. A temperature control zone on the droplet actuator is heated to about 95° C. A sample droplet is dispensed and transported using droplet operations to a detection spot within the 95° C. temperature control zone. Immediately after transport of the sample droplet to the detection spot, fluorescence is measured about every 0.25 seconds. The time for the fluorescence signal to drop to plateau level (i.e., melting is associated with a decrease in fluorescence signal) may be used as an estimate of the minimal PCR denaturation time.

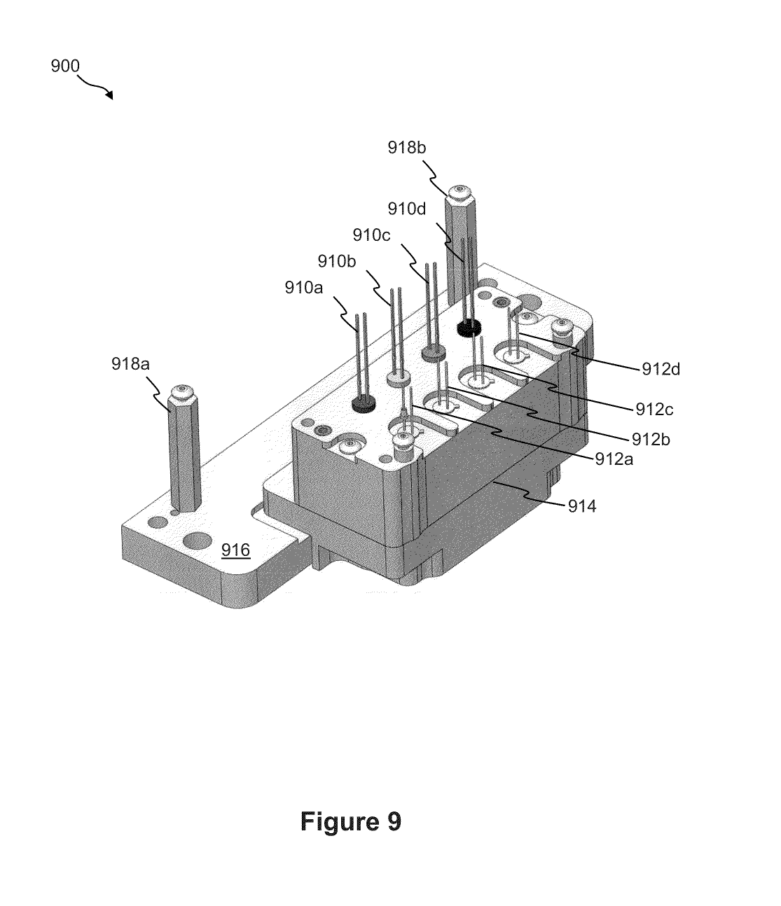

FIG. 3 illustrates a flow diagram of an example of a detection protocol 300 for detection of multiple (e.g., four) fluorophores in a single reaction droplet. Detection of fluorescence signals is performed during the primer/probe annealing step at about 55° C. A detection spot is positioned at a certain droplet operations electrode within the 55° C. temperature control zone of the droplet actuator. The size of the detection spot may be selected to substantially increase fluorescence detection sensitivity. In one example, the detection spot may be smaller in size than the droplet operations electrode such that background fluorescence from the droplet actuator is reduced. Because background fluorescence is reduced, sensitivity of detection is increased. The 2× reaction droplet is positioned at the detection spot for detection of the fluorescent signals. In one example, the fluorimeter is a 4-channel detection system such as the detection system described in reference to FIGS. 9, 10, and 11. Detection protocol 300 may include, but is not limited to, the following steps.

In one step, a droplet, such as the 2× reaction droplet described in reference to FIGS. 1 and 2, is transported to a detection spot on a droplet actuator for real-time detection during PCR amplification.

In another step, voltage is set for operation of a four channel detection system. In one example, the four channel detection system is designed to measure fluorescence from FAM, HEX, TR and Cy5 fluorophores. FAM means 6-carboxyfluorescein. HEX means hexadecimal code to graph the data point obtained in fluorimeter detect operations.

In other steps, FAM, HEX, TR and Cy5 fluorescence signals are measured. After acquisition of fluorescence signals, voltage is returned to pre-operation levels.

In another step, the 2× reaction droplet is transported using droplet operations from the detection spot in the 55° C. temperature control zone to the 72° C. temperature control zone. PCR thermal cycling may continue as described in reference to FIG. 2.

### 7.1.2 Fluorescent Probe Hybridization-Based Amplicon Detection

Probe hybridization is a detection method which measures the hybridization of a labeled probe, e.g., a fluorescently-labeled probe, to a specific amplified DNA sequence (e.g., internal control sequence, target DNA of interest). In one example, the labeled probe may be an oligonucleotide probe that is labeled with a fluorescent reporter molecule and a quencher molecule (i.e., internal FRET probe). The internal FRET probe may, for example, include a fluorophore covalently attached to the 5′-end of the oligonucleotide probe and a quencher molecule at the 3′-end. As long as the fluorophore and the quencher are in proximity, quenching inhibits any fluorescence signals. The exonuclease activity (5′ to 3′) of Taq polymerase is used to degrade the internal FRET probe bound to a specific target sequence. Upon separation of the fluorescent reporter molecule from the quencher molecule, a fluorescent signal is generated that is proportional to the concentration of the target DNA. Internal FRET probes may be used for real-time or endpoint PCR analysis. For end-point analysis, the accumulation of a signal, e.g., a fluorescence signal, is measured after the amplification of the target sequence is complete. For real-time analysis, the signal is measured while the amplification reaction is in progress.

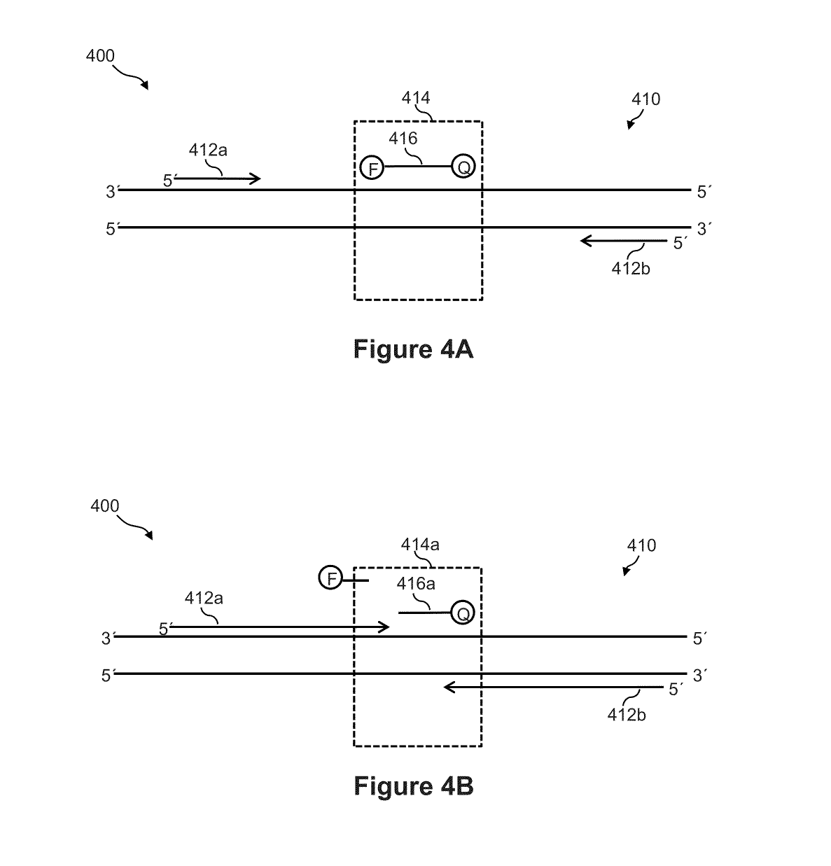

FIGS. 4A and 4B illustrate an example of a process 400 of amplification and probe hybridization for detection of a target sequence. Target sequence 410 may be amplified by PCR using a forward primer 412a and a reverse primer 412b. A probe site 414 may be located between forward primer 412a and reverse primer 412b. While one probe site 414 is shown, any number of probe sites 414 may be located between forward primer 412a and reverse primer 412b. A FRET probe 416 may be designed to anneal to a specific sequence within probe site 414. Referring to FIG. 4B, as forward primer 412a is extended by a polymerase enzyme (e.g., Taq polymerase; not shown), probe 416 is degraded by the exonuclease activity of the enzyme and the fluorophore (F) is separated from the quencher (Q). Because the fluorophore is separated from the quencher, a fluorescent signal is produced.

7.1.3 Fluorophore/Quencher Selection for Multiplexed qPCR

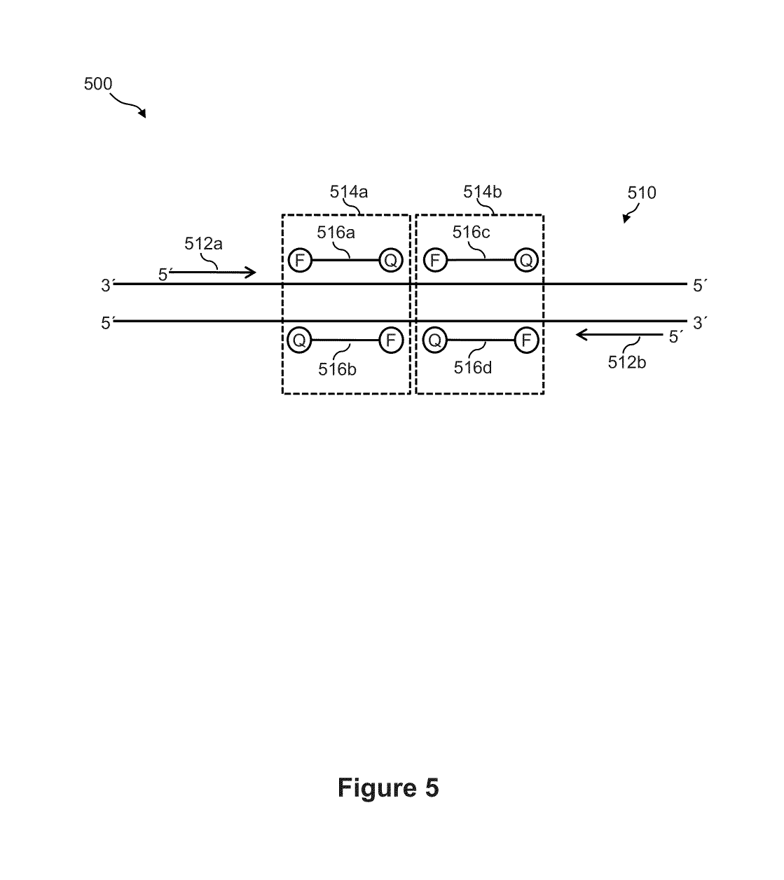

Fluorophore/quencher pairs may be selected for compatibility with the oil filled environment of a droplet actuator. To evaluate various fluorophores/quencher pairs, quantitative real-time PCR (qPCR) amplification of a region of the Escherichia coli (E. coli) polymerase III subunit α gene was performed on a droplet actuator. Forward and reverse primers were selected to amplify a 109 bp fragment of the E. coli polymerase III subunit α gene. Two probe sites were selected within the 109 bp fragment. FIG. 5 illustrates an example of a primer/probe arrangement 500 for multiplexed PCR amplification of the 109 bp fragment of the E. coli polymerase III subunit α gene. Primer/probe arrangement 500 includes a forward primer 512a and a reverse primer 512b that flank a target sequence 510. Forward primer 512a and a reverse primer 512b are used to amplify target sequence 510. Primer/probe arrangement 510 includes two probe sites 514a and 514b located between forward primer 512a and reverse primer 512b. One or more FRET detection probes 516, e.g., four FRET detection probes 516a through 516d, may be designed to anneal to specific sequences within probe sites 514.

In one example, FRET detection probe 516a may include a Cy5 fluorophore; FRET detection probe 516b may include a FAM fluorophore or a UV fluorophore; FRET detection probe 516c may include a HEX fluorophore; and FRET detection probe 516a may include a Texas Red fluorophore. Examples of suitable combinations of fluorophores for probe sites 514a and 514b are shown in Table 1. Excitation and emission wavelengths of UV, FAM, HEX, Texas Red and Cy5 are shown in Table 2. Examples of suitable fluorophore/quencher pairs are shown in Table 3.

TABLE 1
Fluorophore probe site combinations

| Probe site 514a | Probe site 514b |
|---|---|
| UV | HEX |
| UV | Texas Red |
| FAM | HEX |
| FAM | Texas Red |
| HEX | Cy5 |
| Texas Red | Cy5 |

TABLE 2
Fluorophore excitation and emission wavelengths (nm)

| | Excitation | Emission |
|---|---|---|
| UV (ATTO 390) | 390 | 479 |
| FAM | 494 | 520 |
| HEX | 535 | 556 |
| Texas Red (TR) | 583 | 603 |
| Cy5 | 646 | 662 |

TABLE 3
Fluorophore-quencher pairs

| Fluorophore | Quencher* |
|---|---|
| UV | EDQ |
| FAM | EDQ |
| FAM | BHQ1 |
| HEX | EDQ |
| Texas Red (TR) | DDQ2 |
| Cy5 | BHQ2 |
| Cy5 | DDQ2 |

*EDQ: 3′ Eclipse Dark Quencher; BHQ Black Hole Quencher; DDQ: Deep Dark Quencher

In another example, referring to FIG. 5, FRET detection probe 516a may include an ATTO 647 fluorophore; FRET detection probe 516b may include a FAM fluorophore or a UV fluorophore; FRET detection probe 516c may include a HEX fluorophore; and FRET detection probe 516a may include a TR fluorophore. A summary of probe characteristics including fluorophore/quencher pairs, excitation and emission wavelengths of the fluorophores and probe sequences for this example are shown in Table 4.

TABLE 4
Summary of probe characteristics

| | | Excitation | Emission | | Sequence |
|---|---|---|---|---|---|
| Fluorophore | Quencher | (nm) | (nm) | Sequence | ID |
| ATTO 390 | EDQ | 390 | 479 | ATC CGG GAA GTG TTC TTC | 1F |
| | | | | AT | |
| FAM | BHQ1 | 494 | 520 | ATC CGG GAA GTG TTC TTC | 1F |
| | | | | AT | |
| FAM | EDQ | 494 | 520 | ATC CGG GAA GTG TTC TTC | 1F |
| | | | | AT | |
| HEX | EDQ | 535 | 556 | ACC AGC GCG CTG GTA GAT | 2R |
| | | | | GA | |
| TR | DDQ2 | 583 | 603 | TCA TCT ACC AGC GCG CTG | 2F |
| | | | | TT | |
| Cy5 | BHQ2 | 646 | 662 | AT GAA GAA CAC TTC CCG | 1R |
| | | | | GAT | |
| Cy5 | DDQ2 | 646 | 662 | AT GAA GAA CAC TTC CCG | 1R |
| | | | | GAT | |
| ATTO 647N | DDQ2 | 650 | 665 | AT GAA GAA CAC TTC CCG | 1R |
| | | | | GAT | |

*EDQ: 3′ Eclipse Dark Quencher; BHQ: Black Hole Quencher; DDQ: Deep Dark Quencher

Fluorophore/quencher pairs shown in Table 4 were evaluated on a droplet actuator using real-time PCR (qPCR) amplification of the E. coli polymerase III subunit α. In this example, E. coli DNA was prepared on-bench using the ChargeSwitch® gDNA Mini Bacteria kit (Invitrogen: CS11301) following manufacturer's instructions. PCR primers were designed against the α subunit of polymerase III using the NCBI Blast “pick primers” tool and obtained from IDT DNA (NCBI accession NC_{—}012947; Forward primer: GGTGCGGATCAGCTCGAGAA, Reverse primer: CGACGTCGGACGCAGTCTTTT; Skokie, Ill.). Probes were designed to two sequences between the PCR primers. PCR reactions (8 μL; 1 unit Platinum® Taq DNA polymerase (Invitrogen), 1×PCR buffer, 0.4 mM dNTPs (Roche or Kapa), 1.5 mM MgCl₂, 1.1 μM each forward and reverse primers, 1.3 μM each probe, and about 50 ng of DNA) were prepared on-bench. An aliquot (3.4 μL) of each PCR reaction was loaded onto separate sample reservoirs of a droplet actuator and thermal cycled as follows: 1 minute hot start at 95° C., followed by 45 cycles of 7 seconds at 95° C., 20 seconds at 55° C., and 1 second at 72° C. Cts were calculated using a 6-parameter logistic fit. Due to predictable artifacts in the data, a conventional 5PL fitting technique was modified to produce more consistent Ct values for the data sets provided.

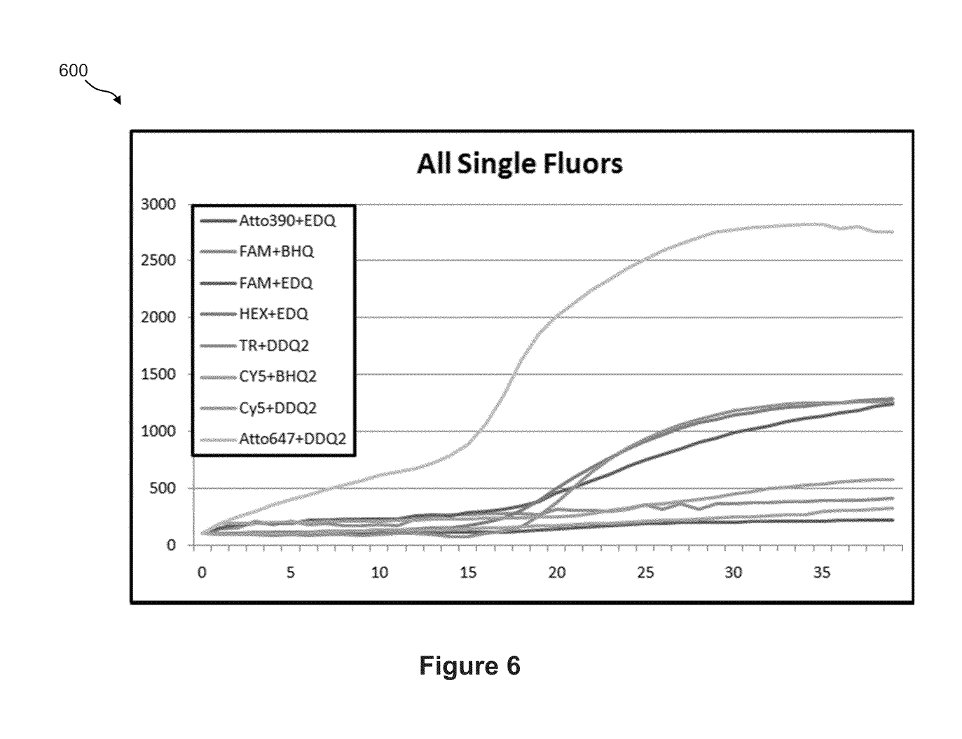

FIG. 6 shows a plot 600, which is an example of a plot of fluorescence data from eight separate qPCR amplifications, each using a single FRET detection probe. The amplification reactions were performed using the primer/probe arrangement described in reference to FIG. 5 and Table 4. Each PCR reaction using a single fluorophore/quencher pair was performed using the same reaction conditions and the same template DNA. qPCR was performed for 40 cycles. The data were adjusted to an initial fluorescence value of 100 to compensate for varying background levels. Some variation in final fluorescence value is evident, ranging from a small increase with the ATTO 390 (UV) probe to a very large increase with the ATTO 647 probe. Because the individual PCR reactions used the same reaction conditions and template DNA, variation in final fluorescence values is likely due to detector sensitivity and/or characteristics of the fluorophore.

A summary of the maximum change in fluorescence of each probe during 40 cycles of PCR is shown in Table 5. ATTO 390 probe had the smallest increase in fluorescence, while ATTO 647 had the largest in fluorescence. Most of the fluorophores (FAM, HEX, and TR) had about the same change in fluorescence. Despite inter-assay variation, the data in Table 5 represents the typical results seen in probe analysis. Comparing EDQ and BHQ quenchers on a FAM-labeled probe did not have a significant impact on the change in fluorescence. In contrast, DDQ2 more than doubled the change in fluorescence compared to BHQ2 on a Cy5-labeled probe. Because of the relatively low fluorescence signal from Cy5-labeled probes, ATTO 647N/DDQ2 fluorophore/quencher pair may be used to provide a significant increase in fluorescence signal (i.e., about a 5.5 fold increase). The data demonstrates the variations in fluorescence signal provided by different fluorophore labeled probes during qPCR on a droplet actuator. Fluorophore/quencher pairs may be selected for specific microfluidic systems to optimize signal amplification.

TABLE 5
Maximum change in fluorescence

| Probe | Quencher | Δ Fluorescence |
|---|---|---|
| ATTO 390 | EDQ | 119 |
| FAM | EDQ | 1144 |
| FAM | BHQ1 | 1113 |
| HEX | EDQ | 1192 |
| TR | DDQ2 | 1163 |
| Cy5 | BHQ2 | 221 |
| Cy5 | DDQ2 | 481 |
| ATTO 647N | DDQ2 | 2655 |

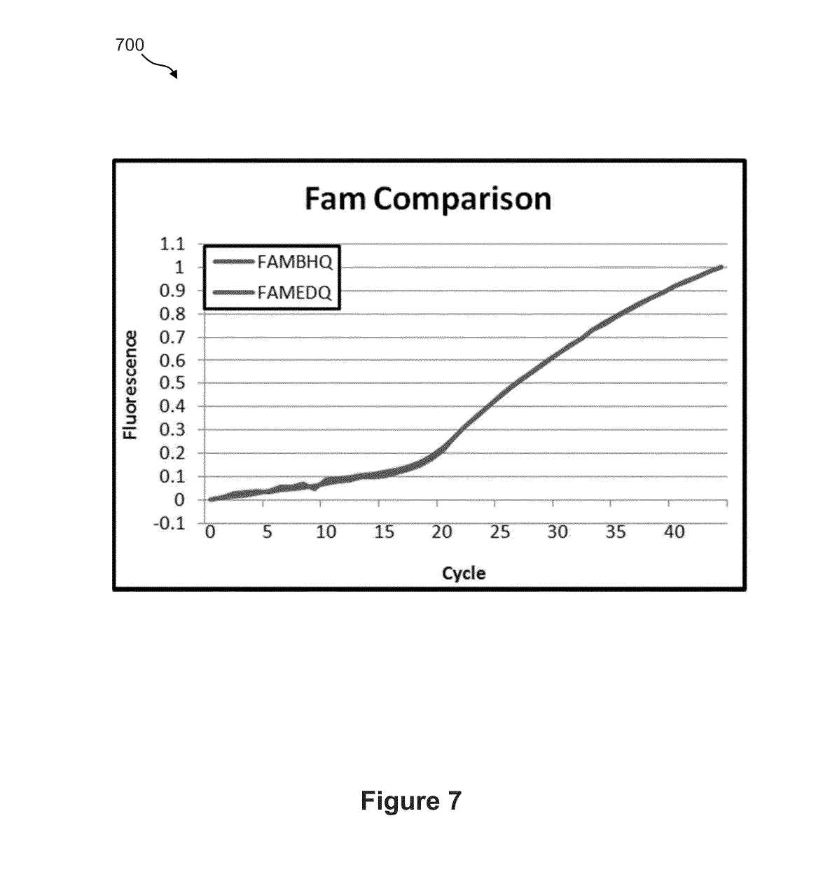

FIG. 7 shows a plot 700, which is an example of a plot of fluorescence data of a comparison of fluorophore/quencher pair on probe properties during qPCR amplification. In this example, two separate qPCR amplifications were performed. In one reaction, the detection probe combination was FAM/BHQ and in the other reaction the detection probe combination was FAM/EDQ. The reactions were run side by side on the droplet actuator and evaluated under different detectors. To remove any detector-specific affects, the data were zeroed and normalized on a scale from 0 to 1 (all data subtracted by first point of data and divided by final point of data). Comparison of the two quenchers (BHQ and EDQ) in this example indicates that there is no difference in signal between the two probes on-actuator.

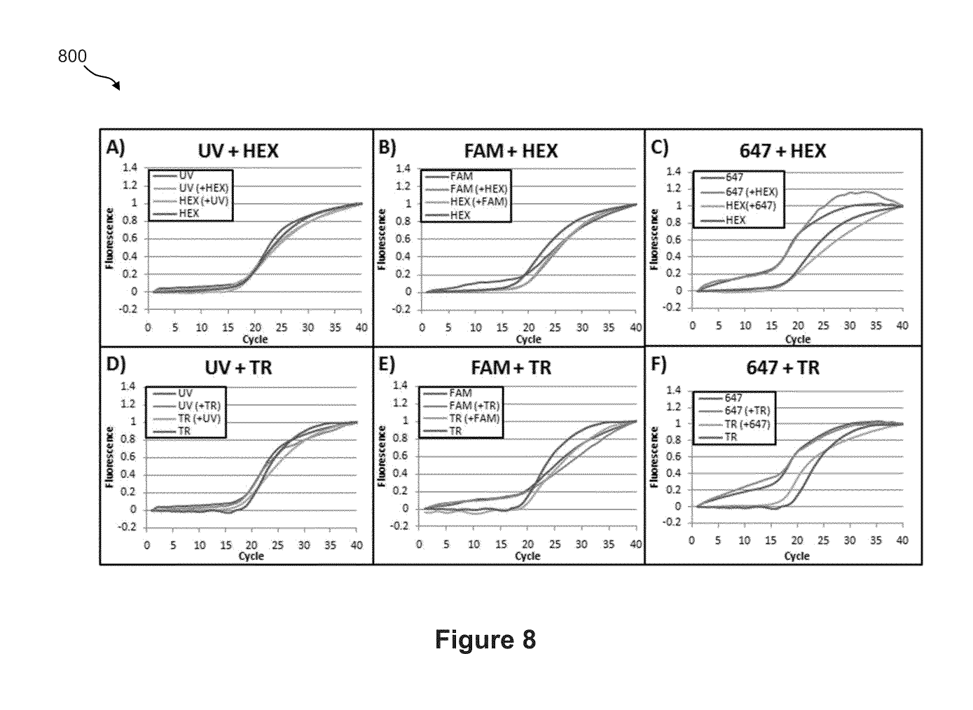

FIG. 8 shows plots 800A through 800F, which are example of plots of fluorescence data from qPCR amplifications comparing single probe and double probe (two-plex) reactions. For each comparison, three separate qPCR amplifications were performed. Experimental comparisons were made between two reactions each using a single fluorophore labeled probe and a two-plex reaction containing both probes. Data were zeroed and normalized 0 to 1.

Referring to plot 800A, experimental comparisons were made between reactions of single probes containing fluorophore UV or HEX, and a two-plex reaction containing both probes. The two-plex reaction was detected under UV (denoted “UV (+HEX)”) or HEX (denoted “HEX (+UV)”).

Referring to plot 800B, experimental comparisons were made between reactions of single probes containing fluorophore FAM or HEX, and a two-plex reaction containing both probes. The two-plex reaction was detected under FAM (denoted “FAM (+HEX)”) or HEX (denoted “HEX (+FAM)”).

Referring to plot 800C, experimental comparisons were made between reactions of single probes containing fluorophore ATTO 647 or HEX, and a two-plex reaction containing both probes. The two-plex reaction was detected under ATTO 647 (denoted “ATTO 647 (+HEX)”) or HEX (denoted “HEX (+ATTO 647)”).

Referring to plot 800D, experimental comparisons were made between reactions of single probes containing fluorophore UV or TR, and a two-plex reaction containing both probes. The two-plex reaction was detected under UV (denoted “UV (+TR)”) or TR (denoted “TR (+UV)”).

Referring to plot 800E, experimental comparisons were made between reactions of single probes containing fluorophore FAM or TR, and a two-plex reaction containing both probes. The two-plex reaction was detected under UV (denoted “FAM (+TR)”) or TR (denoted “TR (+FAM)”).

Referring to plot 800F, experimental comparisons were made between reactions of single probes containing fluorophore ATTO 647 or TR, and a two-plex reaction containing both probes. The two-plex reaction was detected under ATTO 647 (denoted “ATTO 647 (+TR)”) or TR (denoted “TR (+ATTO 647)”).

The data curves were zeroed and normalized to clarify differences in crossing threshold (Ct) without initial background and to remove complicating factors such as differences in fluorophore fluorescence as describe in reference to Table 5. Initial cycling qPCR curves for ATTO 647 in plots 800C and 800F indicate background probe hydrolysis. Amplification curves generally maintained the same curvature in a two-probe reaction as in a single-probe reaction. The data suggest that there is no significant interference when multiple probes are used, compared to the single probe reactions, as determined by a visual Ct and curvature comparison.

7.1.4 Comparison of On-Bench and On-Actuator qPCR

An example of a comparison of Ct values obtained for single and multiplexed qPCR performed on-bench and on-actuator are shown in Table 6. In this example, E. coli DNA was prepared using the ChargeSwitch® gDNA Mini Bacteria kit (Invitrogen: CS11301) following manufacturer's instructions. Forward and reverse primers and probe arrangements were as described in reference to FIG. 5. qPCR reaction conditions for on-actuator amplifications were as described in reference to FIGS. 6, 7, and 8. On-bench qPCR reactions (25 μL) were made with 1 unit Platinum® Taq DNA polymerase (Invitrogen), 1×PCR buffer, 0.2 mM dNTPs (Roche or Kapa), 1.5 mM MgCl₂, 0.2 μM each forward and reverse primers, 0.15 μM each probe, and about 250 ng of E. coli DNA. Reactions were run on a Roche LC480 droplet actuator instrument with the following thermal cycles: 2 minute hot start at 95° C., followed by 45 cycles of 15 seconds at 95° C. and 40 seconds at 55° C.

TABLE 6
Comparison of Ct values for single- and two-probe qPCR reactions
performed on-bench and on-actuator

| | | Probe 1 | | Probe 2 | |
|---|---|---|---|---|---|
| Probe 1 | Probe 2 | Bench | Actuator | Bench | Actuator |
| ATTO 390 | — | N/A* | 18.19 | — | — |
| FAM | — | 17.36 | 24.62 | — | — |
| HEX | — | 17.12 | 17.53 | — | — |
| TR | — | 16.67 | 18.72 | — | — |
| ATTO 647N | — | 14.63 | 12.86 | — | — |
| ATTO 390 | HEX | N/A | 17.095 | 15.82 | 16.709 |
| ATTO 390 | TR | N/A | 17.686 | 15.13 | 17.758 |
| FAM | HEX | 17.17 | 19.394 | 17.03 | 19.061 |
| FAM | TR | 17.22 | 20.025 | 16.34 | 18.791 |
| ATTO 647N | HEX | 14.46 | 11.933 | 15.84 | 16.045 |
| ATTO 647N | TR | 14.79 | 16.454 | 15.34 | 16.845 |

*N/A: on-bench Roche LC480 does not have an applicable filter to measure ATTO 390

### 7.2 Detection System

The detection system of the present invention uses a single excitation beam and a single detection beam to collect multiple (e.g., four) different fluorescent signals at a single detection spot on a droplet actuator. By using an arrangement of light-emitting diodes (LEDs) that includes excitation lenses, filters, and mirrors, multiple (e.g., four) different excitation wavelengths are multiplexed in a single excitation beam. Similarly, by using an arrangement of detectors (e.g., photodiodes) that includes mirrors, filters and detection lenses, a single optical beam is used for detection of the corresponding emission signals.

FIGS. 9, 10 and 11 illustrate various views of an example a detection system 900 for detection of multiple (e.g., four) different fluorophores in a sample droplet at a single detection spot. Referring to FIG. 9, which is a perspective view of detection system 900, detection system 900 may include multiple excitation LEDs 910, e.g., LEDs 910a through 910d. Detection system 900 may also include multiple detectors 912, e.g., detectors 912a through 912d. Detection system 900 may be enclosed in a housing 914. Housing 914 may be attached to a base plate 916. Base plate 916 is used to position detection system 900 in the microfluidics system (not shown) that is holding a droplet actuator. A set of mounting pins 918 may be used to position detection system 900 in the microfluidics system (not shown). Detection system 900 is positioned such that a single excitation beam of light and a single detection beam are directed to a detection spot (not shown) on a droplet actuator.

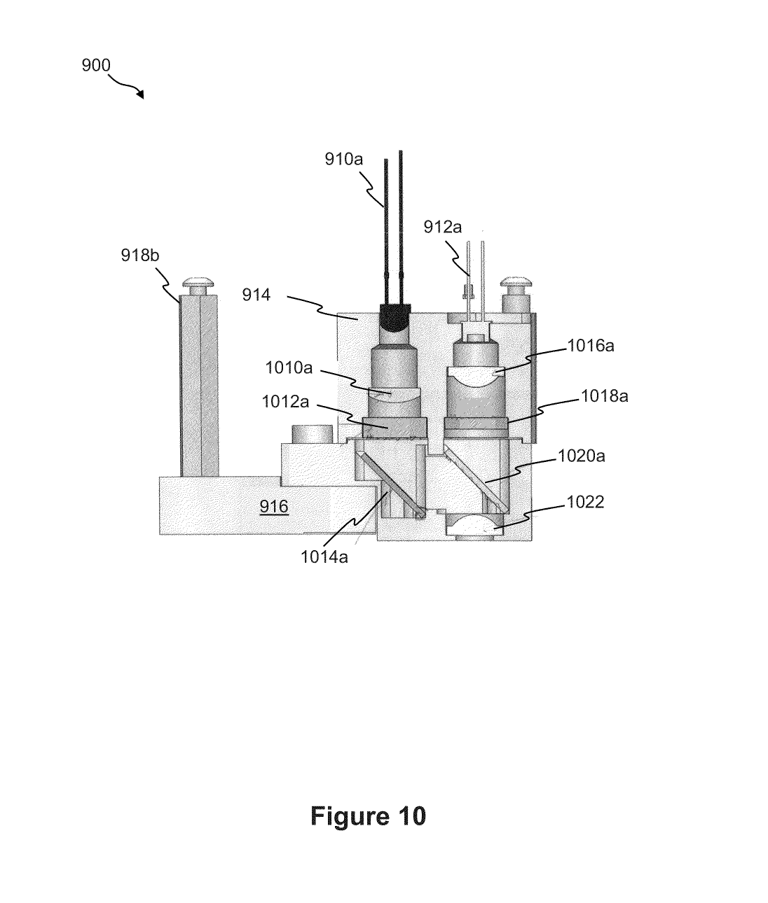

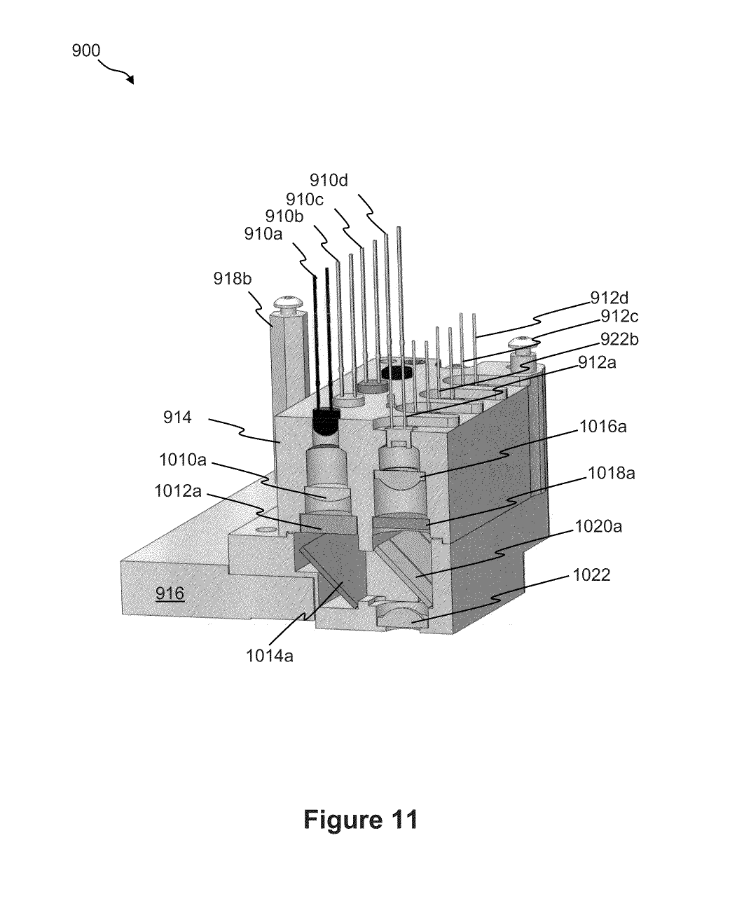

Referring to FIGS. 10 and 11, which are cross-sectional views of detection system 900, excitation LEDs 910 are aligned with multiple excitation lenses 1010, multiple excitation filters 1012, and multiple mirrors 1014, respectively. By way of example, detection system 900 may include four LEDs 910a through 910d that are aligned with four excitation lenses 1010a through 1010d, four excitation filters 1012a through 1012d, and four mirrors 1014a through 1014d, respectively. Each excitation lens 1010 is used to focus light emitted from its corresponding LED 910 onto the corresponding excitation filter 1012. Each excitation filter 1012 is used to select a certain wavelength of light emitted from its corresponding LED 910 that may be used for excitation of a certain fluorophore. Each mirror 1014 is used to direct the filtered light (i.e., light of a certain wavelength) to a corresponding second directing mirror that is positioned in proximity of a corresponding excitation focusing lens. Each directing mirror and corresponding focusing lens is used to multiplex the different wavelengths of light (e.g., four different wavelengths of light) into a single excitation beam.

Detectors 912 are aligned with multiple detection lenses 1016, multiple detection filters 1018, and multiple selective dichroic filters 1020, respectively. By way of example, detection system 900 may include four detectors 912a through 912d that are aligned with four detection lenses 1016a through 1016d, four detection filters 1018a through 1018d, and four dichroic filters 1020a through 1020d, respectively. Each dichroic filter 1020 is used to select a certain wavelength of light corresponding to the emission wavelength of a certain fluorophore. A focusing lens 1022 and a directing mirror (not shown) are used to multiplex different wavelengths of light emitted from one or more fluorophores (e.g., four different fluorophores) into a single detection beam.

A suitable detection system is described in U.S. Provisional Patent App. No. 61/361,576, the entire disclosure of which is incorporated herein by reference.

Examples of fluorophore combinations suitable for two examples of four-channel detection systems are shown in Tables 7 and 8.

TABLE 7
Detection System 1: Cy5, HEX, BBT*, UV

| | Red | Green | Blue | UV |
|---|---|---|---|---|
| Example | Cy5, ATTO 647 | HEX | BBT | 4MU, ATTO 390 |
| fluorophore | | | | |
| Excitation (nm) | 608-648 | 532-554 | 387-447 | 352-402 |
| Emission (nm) | 672-712 | 573-613 | 542-582 | 417-477 |

*BBT is a product of enzymatic reaction with AttoPhos substrate.

TABLE 8
Detection System 2 Cy5, Texas Red, HEX, FAM

| | Red | Yellow | Green | Fam |
|---|---|---|---|---|
| Example | Cy5, | Texas Red (TR) | HEX | FAM, FITC |
| fluorophore | ATTO 647 | | | |
| Excitation (nm) | 608-648 | 568-592 | 532-554 | 458-492 |
| Emission (nm) | 672-712 | 608-648 | 573-613 | 509-551 |

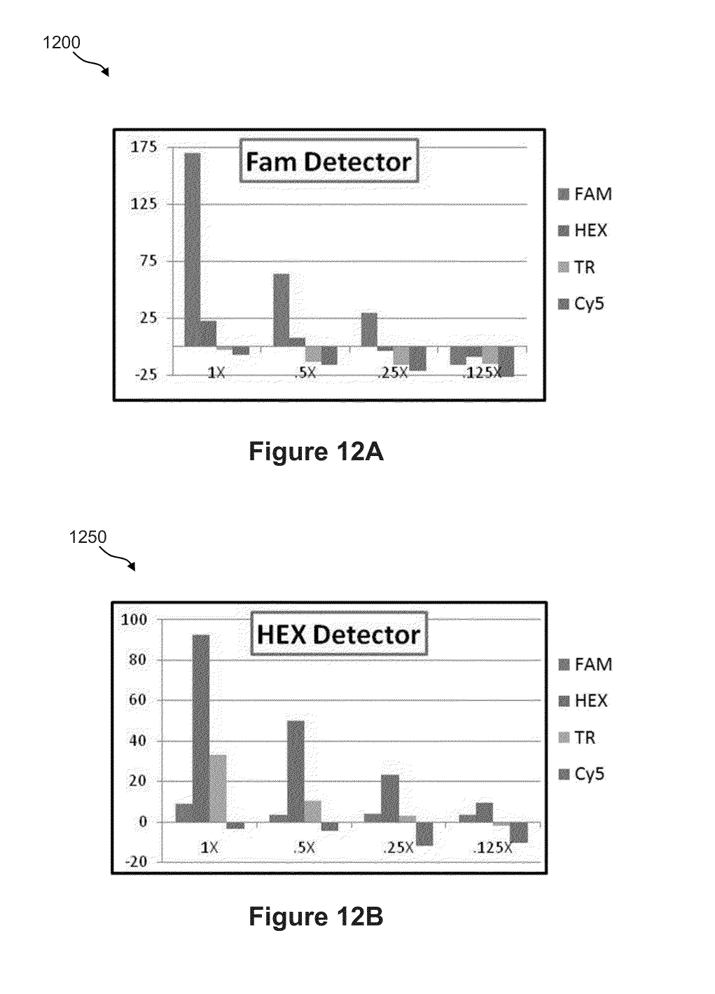

FIGS. 12A and 12B show bar graphs 1200 and 1250, respectively, which are examples of bar graphs of FAM and HEX fluorophore calibration data obtained on-actuator. To test the detectors of detection system 900 of FIGS. 9, 10, and 11 for spectral overlap of FAM and HEX fluorophores, a droplet containing a single type of fluorophore dye, i.e., FAM or HEX, was transported to the detection spot of a droplet actuator and fluorescence measured. Each fluorescent dye was analyzed using each detector (e.g., detectors 912a through 912d).

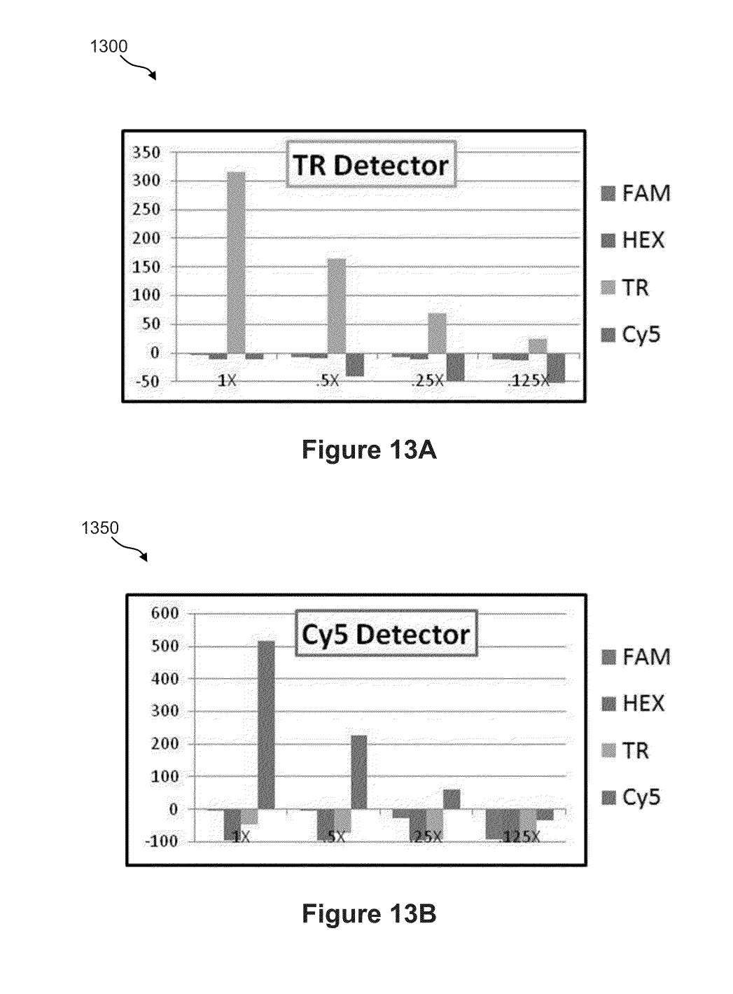

FIGS. 13A and 13B show bar graphs 1300 and 1350, respectively, which are examples of bar graphs of TR and Cy5 fluorophore calibration data obtained on-actuator. To test the detectors of detection system 900 of FIGS. 9, 10, and 11 for spectral overlap of TR and Cy5 fluorophores, a droplet containing a single type of fluorophore dye, i.e., TR or Cy5, was transported to the detection spot of a droplet actuator and fluorescence measured. Each fluorescent dye was analyzed using each detector (e.g., detectors 912a through 912d).

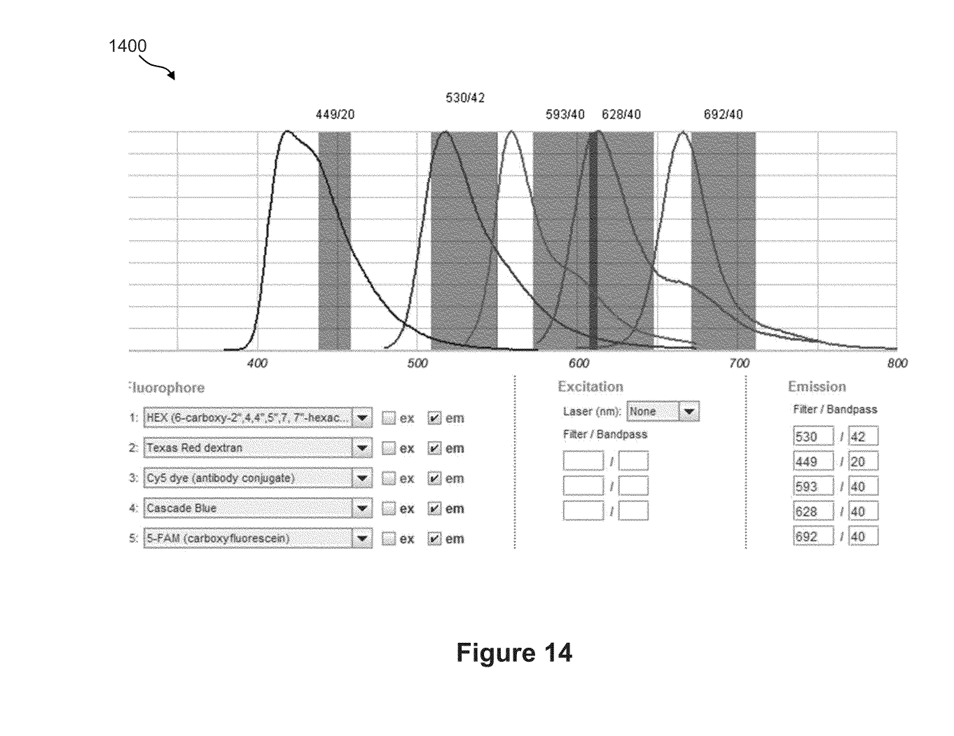

FIG. 14 shows a screenshot 1400 of an example of an overlay of a multiplexed fluorophore emission and detection spectra. The shading in screenshot 1400 represents spectrum covered by detectors for emissions. The long emission tails for each fluorophore may be deconvoluted for multiplexed analysis. Cascade Blue is spectrally similar to ATTO 390.

7.3 Multiplexed qPCR for Detection of Infectious Agents

The present invention provides a droplet actuator device and methods for multiplexed reverse transcription-qPCR (RT-qPCR) detection of an infectious agent in a biological sample. In one embodiment, the methods of the invention provide for multiplexed RT-qPCR detection of one or more types of influenza virus on a droplet actuator. In another embodiment, the methods of the invention provide for multiplexed detection of human immunodeficiency virus (HIV) on a droplet actuator. In a preferred embodiment, the invention provides a droplet actuator device and methods for integrated sample preparation and multiplexed detection of an infectious agent (e.g., HIV, influenza). In another embodiment, sample preparation (i.e., isolation of viral RNA) may be performed on-bench and subsequently loaded onto a fluid dispensing reservoir of a droplet actuator.

The droplet actuator of the invention may be designed to fit onto the deck of a droplet actuator instrument that houses additional droplet actuator features, such as one or more magnets for immobilization of magnetically responsive beads, one or more heater assemblies (e.g., 3 heater assemblies) for controlling the temperature within certain reaction and/or washing zones, and a fluorescence detection system. In one example, an ESElog fluorimeter (available from Qiagen) may be mounted onto the droplet actuator instrument using Thor lab 30 mm cage system. Commercially available fluorimeter configurations (Catalog number: 9002063; E1:365 nm/D1:460 nm and E2:520 nm/D2: 570 nm) and custom configurations (E1:470 nm/D1:520 nm and E2:545 nm/D2: 620 nm) may be use to simultaneously detect fluorescence within a single droplet at one location using, for example, confocal optics. In a preferred embodiment, the custom configuration of the fluorimeter is used. The fluorimeter may be connected to a computer which also interfaces with the droplet actuator instrument via a USB connection. In one example, the connection between the fluorimeter and the droplet actuator instrument is not automated. Because the connection between the fluorimeter and the droplet actuator instrument is not automated, each fluorescent reading must be taken manually. In another example, the connection between the fluorimeter and the droplet actuator instrument is automated and fluorescent readings are automatically acquired.

The droplet actuator may be filled with a filler fluid, such as silicone oil, to prevent evaporation of the droplets and to facilitate droplet operations. In one example, the filler fluid may be polydimethylsiloxane oil (7 cSt polydimethylsiloxane (Gelest) with 0.005% Span 85). The oil filler fluid may be degassed to minimize the appearance of bubbles prior to loading into the gap of a droplet actuator. For example, the oil may be vacuum-degassed for about 5 hours and stored under vacuum until use. When an aliquot of oil is removed from the vacuum chamber, the remaining oil may be degassed for an additional 30 minutes before use. Each aliquot of degassed oil may be assigned a unique lot number and used immediately. Droplet operations (e.g., transport, splitting, merging) within the oil filled environment of the droplet actuator may be controlled by software, e.g., Assay Development Environment software.

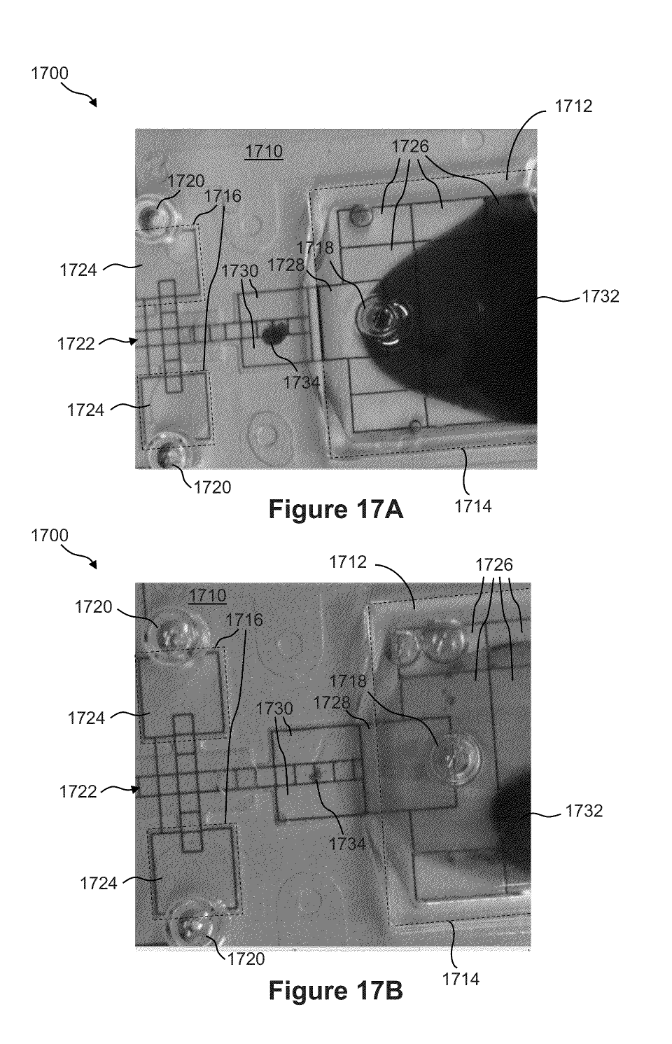

In a preferred embodiment, the invention provides a droplet actuator device and methods for integrated sample preparation and multiplexed detection of an infectious agent (e.g., HIV, influenza). An example, of an integrated sample preparation protocol is described in reference to FIGS. 17A and 17B. In another embodiment, sample preparation (e.g., isolation of viral RNA) may be performed on-bench and subsequently loaded onto a fluid dispensing reservoir of a droplet actuator. In one example, viral RNA may be prepared using a ChargeSwitch Total RNA Kit (Life Technologies).

In one example, a RT-qPCR master mix solution (e.g., about 15 μL to about 20 μL) that includes enzyme mix (i.e., reverse transcriptase and DNA polymerase), McOAc, forward and reverse primers, probes and RNA may be prepared on-bench and loaded onto a fluid reservoir of a droplet actuator. Reagent concentrations and amount of RNA may be selected for specific amplification of target sequences. An example of a RT-qPCR master mix solution composition for two-plex amplification of influenza A (Flu) and bacteriophage MS2 (internal process control) is shown in Table 9. In this example, a HawkZ05 Fast One-Step RT-PCR Kit (Roche: 05987687 190) is used for reverse transcription and amplification of viral RNA.

TABLE 9
Example RT-qPCR master mix composition for Flu and MS2
amplification

| | | | Final | |
|---|---|---|---|---|
| | Concentration | Units | Concentration | μL/Reaction |
| HawkZ05 | 2.273 | X | 1.365 | 9.0 |
| RT-PCR enzyme | | | | |
| mix | | | | |
| McOAc | 25 | mM | 1.88 | 1.1 |
| Flu forward | 10 | μM | 0.25 | 0.375 |
| primer | | | | |
| Flu reverse | 10 | μM | 0.25 | 0.375 |
| primer | | | | |
| MS2 forward | 10 | μM | 0.25 | 0.375 |
| primer | | | | |
| MS2 reverse | 10 | μM | 0.25 | 0.375 |
| primer | | | | |
| Flu probe | 10 | μM | 0.2 | 0.3 |
| MS2 probe | 10 | μM | 0.2 | 0.3 |
| Flu RNA | | | | 1.4 |
| MS2 RNA | | | | 1.4 |

| | |
|---|---|
| Reaction Total | 15 μL |

An example of an on-actuator protocol for multiplexed detection of infectious agents (e.g., influenza, HIV) includes, but is not limited to, the following steps: A volume (e.g., about 2 mL) of oil filler fluid (e.g., 7 cSt polydimethylsiloxane with 0.005% Span 85) is loaded into the gap of a droplet actuator through a fluid reservoir (e.g., a sample reservoir). Heater bars on the droplet actuator instrument deck are set to three different temperatures (e.g., 55° C., 60° C., and 65° C.) to provide three temperature control zones on the droplet actuator. Load RT-qPCR master mix (e.g., about 15 μL) solution containing viral RNA onto a fluid dispensing reservoir of the droplet actuator. The RT-qPCR master mix solution may be prepared on-bench as described in reference to Table 9. Dispense one 2× sample droplet from a sample fluid dispensing reservoir. For the reverse transcriptase hot start reaction, transport the 2× sample droplet using droplet operations to the 55° C. temperature control zone and incubate for 150 seconds. Transport the 2× sample droplet using droplet operations to the 60° C. temperature control zone and incubate for 150 seconds. Transport the 2× sample droplet using droplet operations to the 65° C. temperature control zone and incubate for 150 seconds. Reset the heater bars to three different temperatures (e.g., 95° C., 72° C., and 60° C.) to provide three temperature control zones on the droplet actuator for qPCR amplification. Thermal cycle (e.g., about 40 to about 50 cycles) the 2× sample droplet as follows: 5 seconds at 92° C. for denaturation, 1 second at 72° C., and 15 seconds at 60° C. for annealing and extension. Detection is performed by fluorescence measurement at the end of every annealing/extension cycle at the 60° C. detection electrode. The fluorimeter (e.g., ESElog fluorimeter) settings may, for example, be set at the highest sensitivity and dark corrected. Both excitation and detection channels may be turned on simultaneously. The raw data of a real-time PCR run may be processed using Advanced Liquid Logic's Ct calculator program to calculate the threshold cycle number. The program is based on a modified sigmoid model which can tolerate non-ideal issues such as baseline drift and lack of plateau phase. A Ct value of >40 is assigned a “No Ct” value.

In another example, three-plex RT-qPCR amplification was used to detect influenza A (FluA) and influenza B (FluB). MS2 viral RNA was used as an internal control. FluA, FluB and MS2 phage were obtained from Zeptometrix (NATFLUAH1-ST, NATFLUB-ST, #0810052; MA). FluA primers and probe sequences were according to the CDC¹. Forward (5′-GACCRATCCTGTCACCTCTGAC-3′) and reverse (5′-AGGGCATTYTGGACAAAKCGTCTA-′3) primers were obtained from Integrated DNA Technologies, Inc (IDT). Probe (5′-FAM TGCAGTCCTCGCTCACTGGGCACG BHQ1-3′) was obtained from Sigma. FluB primers and probe were designed against a nonstructural protein gene². Forward (5′-TCCTCAACTCACTCTTCGAGCG-3′) and reverse (5′-CGGTGCTCTTGACCAAATTGG-3′) primers were obtained from IDT. Probe (5′-ATTO647N CCAATTCGAGCAGCTGAAACTGCGGTG DDQ2-3′) was obtained from Eurogentec. MS2 primers and probe were designed against the replicase coding sequence³. Forward (5′-GCTCTGAGAGCGGCTCTATTG-3′) and reverse (5′-CGTTATAGCGGACCGCGT-3′) primers were obtained from IDT. Probe (5′-TexasRed CCGAGACCAATGTGCGCCGTG DDQ2-3′) was obtained from Eurogentec.

Influenza A (FluA), influenza B (FluB) and MS2 viral RNAs were isolated separately on-bench or as a combined sample of MS2 spiked into FluA and/or FluB, using a modified protocol with reagents from the ChargeSwitch® Total RNA kit (Invitrogen: CS14010). Briefly, a viral sample (FluA, FluB, MS2, and/or spiked samples) was diluted into viral Universal Transport Medium (UTM; Copan Diagnostics) to a final volume of 200 μl. The diluted viral sample was mixed with 300 μL of lysis mix (lysis buffer plus Proteinase k). Samples were incubated for 15 minutes at 60° C. and then briefly held on ice. A 30 μL aliquot of well-mixed ChargeSwitch beads was added to each sample along with 140 μL of binding buffer. Samples were washed twice with 400 μL of wash buffer 13, and subsequently washed twice with 400 μL of wash buffer 14. RNA was eluted from the ChargeSwitch beads with 60 μL of elution buffer.

Samples were reverse transcribed and amplified on-bench or on-actuator. On-bench RT-qPCR amplification was performed using HawkZ05 Fast One-Step RT-PCR Kit (Roche: 05987687 190) following manufacturer's protocol. Each 20 μL RT-qPCR reaction consisted of 1× HawkZ05, 1.5 mM McOAc, 0.2 μM each forward and reverse primer, 0.15 μM each probe and the appropriate RNA (8-20 ng). Reactions were run on a Roche LC480 droplet actuator instrument with the following thermal cycles: 5 minute incubations at 55° C., 60° C. and 65° C. followed by 50 cycles of 5 seconds at 92° C., 40 seconds at 60° C. and 1 second at 72° C. On-actuator RT-qPCR amplification was also performed using the HawkZ05 Fast One-Step RT-PCR Kit with the following changes: 15 μL reactions consisted of 1.365× HawkZ05, 1.88 mM McOAc, 0.25 μM each forward and reverse primer, 0.2 μM each probe and 6 ng RNA. An aliquot (about 3.4 μL) of each reaction was loaded onto separate sample reservoirs of a droplet actuator and thermal cycled as follows: 5 minute incubations at 55° C., 60° C. and 65° C. followed by 50 cycles of 5 seconds at 92° C., 30 seconds at 60° C. and 1 second at 72° C.

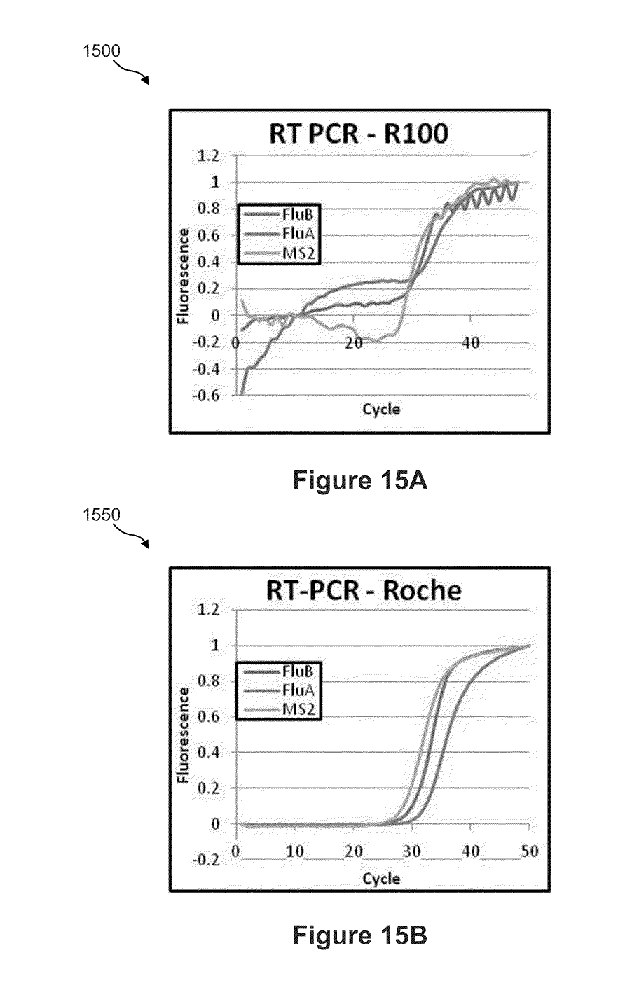

FIGS. 15A and 15B show plots 1500 and 1550, respectively, which are examples of plots of fluorescence data from multiplexed RT-qPCR amplification of influenza A, influenza B, and MS2 (internal process control) generated on-actuator and on-bench, respectively. A master mix solution containing 3 different sets of primers, probes, and RNA (i.e., primers, probes, and RNA for FluA, FluB, and MS2) was aliquoted and amplified simultaneously on-bench and on a droplet actuator. Data were zeroed (i.e., at cycle 10 for on-actuator amplification and at cycle 0 for on-bench amplification) and normalized. Influenza A and Influenza B were amplified along with an internal process control, the MS2 bacteriophage. In both on-actuator and on-bench amplifications, a single reaction was sequentially detected with the 3 relevant filters (FluA: FAM, MS2: TR, FluB: ATTO647). Although more noise was detected in the on-actuator amplified samples, there was no apparent loss in amplification efficiency, as determined by a visual analysis of Ct. Ct values are shown in Table 10.

TABLE 10
Ct values of data in FIGS. 15A and 15B

| Probe | On-Bench | On-Actuator |
|---|---|---|
| FluA | 31.82 | 31.33 |
| FluB | 30.09 | 29.58 |
| MS2 | 28.43 | 27.18 |

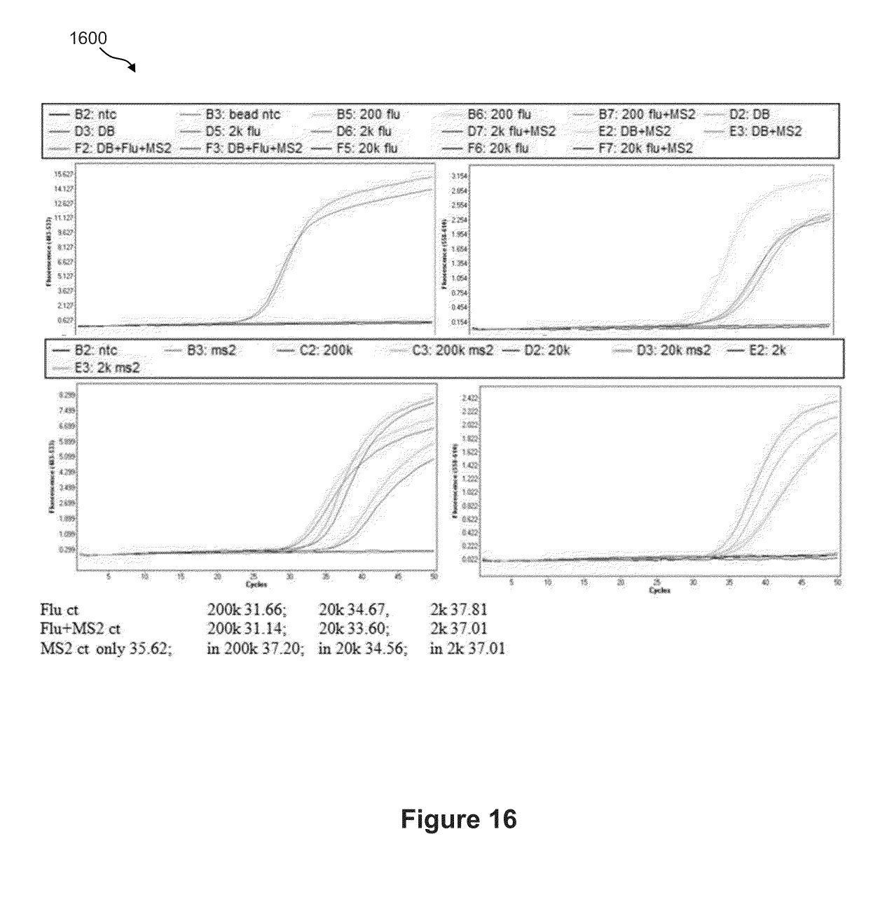

FIG. 16 shows a plot 1600, which is an example of a plot of fluorescence data from multiplexed RT-qPCR amplification of influenza A and MS2 (internal control) in a human sample matrix. In this example, influenza A and MS2 were spiked into a nasal swab media (human sample matrix). RNA was extracted and RT-qPCR was performed on-bench as describe in reference to FIG. 15.

### 7.4 Integrated Sample Preparation and Multiplexed PCR

The present invention provides a droplet actuator device and methods for integrated sample preparation and multiplexed quantitative real-time PCR (qPCR) of nucleic acid targets in a biological sample. Using digital microfluidics technology, the droplet actuator device and methods of the invention provide the ability to perform sample preparation (e.g., nucleic acid isolation and concentration) and multiplexed qPCR from a sample on the same droplet actuator. In various embodiments, the methods of the invention use magnetically response nucleic acid capture beads (e.g., ChargeSwitch beads) for isolation of nucleic acid (e.g., RNA) from a whole blood sample. In one embodiment, the droplet actuator device and methods of the invention may be used for integrated sample preparation and multiplexed RT-qPCR of infectious agents, such as HIV and influenza, in a whole blood sample. Because contaminating components in the matrix of a lysed whole blood sample may “mask” the magnetism of magnetically responsive nucleic acid capture beads, intermediate purification steps may be used to substantially increase the efficiency of bead capture and reduce sample loss. In one example, a whole blood sample preparation protocol may include an agglutination step to remove contaminating cellular debris and particulates. In another example, a whole blood sample preparation protocol may include an elution and re-binding step prior to a final sequence of bead washing to reduce contaminants.

FIGS. 17A and 17B illustrate top views of a portion of an example of a droplet actuator 1700 and show a process of dispensing and concentrating magnetically responsive capture beads in a droplet of whole blood lysate and plasma-like (agglutinated) lysate, respectively. Droplet actuator 1700 may include a bottom substrate 1710 that is separated from a top substrate 1712 by a gap (not shown). Droplet actuator 1700 may also include a sample reservoir 1714 and multiple reagent reservoirs 1716, which are example of on-actuator reservoirs for holding certain fluids in the gap between bottom substrate 1710 and top substrate 1712. Certain features that are integrated into bottom substrate 1710 and/or top substrate 1712 are used to define the boundaries and/or volumes of sample reservoir 1714 and the multiple reagent reservoirs 1716. Sample reservoir 1714 may be loaded with liquid via an input port 1718 in top substrate 1712. Similarly, reagent reservoirs 1716 may be loaded with liquid via respective input ports 1720 in top substrate 1712.

An electrode arrangement on bottom substrate 1710 may include a path, line, and/or array of droplet operations electrodes 1722 (e.g., electrowetting electrodes). Droplet operations electrodes 1722 are arranged with respect to sample reservoir 1714 and the multiple reagent reservoirs 1716. Each of the multiple reagent reservoirs 1716 has a reagent dispensing electrode 1724 that is arranged with respect to the path, line, and/or array of droplet operations electrodes 1722. The dispensing electrode that is associated with sample reservoir 1714 is segmented into an arrangement of multiple individually controlled electrodes, which are arranged with respect to the path, line, and/or array of droplet operations electrodes 1722.

In one example, the multiple individually controlled electrodes of sample reservoir 1714 includes multiple segmented reservoir electrodes 1726. One particular segmented reservoir electrode 1726 of sample reservoir 1714 is arranged in relation to a priming electrode 1728. For example, a certain segmented reservoir electrode 1726 may be arranged in relation to three sides of priming electrode 1728. Priming electrode 1728 is arranged in relation to the path, line, and/or array of droplet operations electrodes 1722 on which droplets may be dispensed. Additionally, a pair of flanking electrodes 1730 may be arranged on the sides of the droplet operations electrodes 1722 near priming electrode 1728. Droplet operations are conducted atop these various electrodes on a droplet operations surface.

Sample reservoir 1714 that includes the multiple individually controlled electrodes supports an on-actuator reservoir that is designed to perform complex droplet mixing and/or droplet dispensing operations. The multiple individually controlled electrodes of sample reservoir 1714 may, for example, be used to manipulate a large volume of sample fluid (e.g., about 1 mL) that contains a quantity of magnetically responsive beads for processing within droplet actuator 1700. For example, sample reservoir 1714 contains a quantity of fluid 1732. Fluid 1732 may, for example, be a whole blood sample combined with a lysis buffer solution. Fluid 1732 may contain a quantity of magnetically responsive beads (not shown), such as nucleic acid capture beads (e.g., ChargeSwitch beads).

A magnet (e.g., a permanent magnet or electromagnet; not shown) may be located in proximity to (e.g., underneath) certain electrodes. For example, the magnet may be located in proximity to the droplet operations electrodes 1722 that are near flanking electrodes 1730. The magnet may be embedded within the deck that holds droplet actuator 1700 when it is mounted on the droplet actuator instrument (not shown). The magnet (not shown) is positioned in a manner which ensures spatial immobilization of magnetically responsive beads during dispensing protocols.

Droplet actuator 1700 may include certain regions for performing certain process steps. For example, droplet actuator 1700 may include, but is not limited to, a sample dispensing region (e.g., storage and dispensing) associated with sample reservoir 1714 and a droplet operations region (e.g., mixing, incubation, washing, detection) associated with the multiple reagent reservoirs 1716 and certain droplet operations electrodes 1714. Additionally, the height of the gap between bottom substrate 1710 and top substrate 1712 at each region may vary. For example, the gap height at the sample dispensing region (i.e., at sample reservoir 1714) may be greater than the gap height at the droplet operations region (i.e., at reagent reservoirs 1716 and/or along droplet operations electrodes 1722). For example, the gap height at sample reservoir 1714 may be about >3 mm to facilitate storage of larger liquid volumes (e.g., about 1 mL) and ready dispensing of droplets. While the gap height at reagent reservoirs 1716 and/or along droplet operations electrodes 1722 may be about 250-500 μm in order to facilitate, for example, rapid transport, mixing, washing, and/or incubation of one or more droplets.

Referring to FIG. 17A, an example of a process of preparing an RNA sample from a whole blood sample using an intermediate elution and re-binding step may include, but is not limited to, the following steps: A blood sample (e.g., about 200 μL) is combined with a volume of lysis buffer solution (e.g., 480 μL lysis buffer plus 20 μL Proteinase K) in sample reservoir 1714. The combined sample and lysis buffer solution is incubated for a period of time sufficient to yield a lysed cell solution (lysate) that contains released nucleic acids (e.g., RNA). In one example, the combined sample and lysis buffer solution is mixed and incubated for about for about 5 minutes. A quantity of magnetically responsive beads (e.g., 10 μL ChargeSwitch beads; not shown) suspended in binding buffer (e.g., 190 μL) is added to the lysate in sample reservoir 1714 and incubated from about 1 minute to about 5 minutes with mixing. A bead washing protocol such as a merge-and-split wash protocol may be used to remove unbound material, yielding a washed bead-containing droplet substantially lacking unbound material. In one example, a bead washing protocol using 36 μL of wash buffer (e.g., RNase free water) is repeated about 10 times. The purified RNA is eluted from concentrated sample droplet 1734 with elution buffer (e.g., about 12 μL). The eluted RNA contained in the droplet surrounding the magnetically responsive beads is re-bound to the magnetically responsive beads. A bead washing protocol using 36 μL of wash buffer (e.g., RNase free water) is repeated about 10 times. The purified RNA is eluted from concentrated sample droplet 1734 with elution buffer (e.g., about 12 μL). The eluted RNA contained in the droplet surrounding the magnetically responsive beads may then be transported away from the beads for further processing on the droplet actuator, e.g., for execution of a droplet based RT-qPCR PCR amplification protocol (not shown). Alternatively, concentrated sample droplet 1734 containing the magnetically responsive beads with purified RNA thereon may be transported away from the magnet for further processing on the droplet actuator, e.g., for execution of a droplet based RT-qPCR amplification protocol using RNA bound to beads (not shown).

Referring to FIG. 17B, an example of a process of preparing an RNA sample from a whole blood sample using an agglutinate may include, but is not limited to, the following steps: A blood sample (e.g., about 200 μL) is combined with a volume of lysis buffer solution (e.g., 480 μL lysis buffer plus 20 μL Proteinase K) and a volume of agglutinate (e.g., 20 μL) in sample reservoir 1714. In one example, the agglutinate may be erythroagglutinin PHA-E that is used to remove cellular debris and particulates. The combined sample and lysis buffer solution is incubated for a period of time sufficient to yield a lysed cell solution (lysate) that contains released nucleic acids (e.g., RNA). In one example, the combined sample and lysis buffer solution is incubated for about 2 minutes for agglutination and then mixed for about 5 minutes. A quantity of magnetically responsive beads (e.g., 10 μL ChargeSwitch beads; not shown) suspended in binding buffer (e.g., 190 μL) is added to the lysate in sample reservoir 1714 and incubated from about 1 minute to about 5 minutes with mixing. A bead concentrating and dispensing protocol (e.g., a bead snap-off protocol) is used to dispense the magnetically responsive beads in a single concentrated sample droplet 1734 on a certain droplet operations electrode 1722 between flanking electrodes 1730, which is near the magnet (not shown). Prior to dispensing concentrated sample droplet 1734, the electrode arrangement on bottom substrate 1710 may be flooded with wash buffer to facilitate dilution and microfluidic operations. Once the concentrated sample droplet 1734, which includes magnetically responsive beads, is formed, the concentrated sample droplet 1734 may be subjected to other droplet operations within droplet actuator 1700. For example, a bead washing protocol, such as a merge-and-split wash protocol, may be used to remove unbound material, yielding a washed bead-containing droplet substantially lacking unbound material (not shown). In one example, a bead washing protocol using 36 μL of wash buffer (e.g., RNase free water) may be repeated from about 10 to about 30 times. The purified RNA may be eluted from concentrated sample droplet 1734 with elution buffer (e.g., about 12 μL). The eluted RNA contained in the droplet surrounding the magnetically responsive beads may then be transported away from the beads for further processing on the droplet actuator, e.g., for execution of a droplet based RT-qPCR PCR amplification protocol (not shown). Alternatively, concentrated sample droplet 1734 containing the magnetically responsive beads with purified RNA thereon may be transported away from the magnet for further processing on the droplet actuator, e.g., for execution of a droplet based RT-qPCR amplification protocol using RNA bound to beads (not shown).

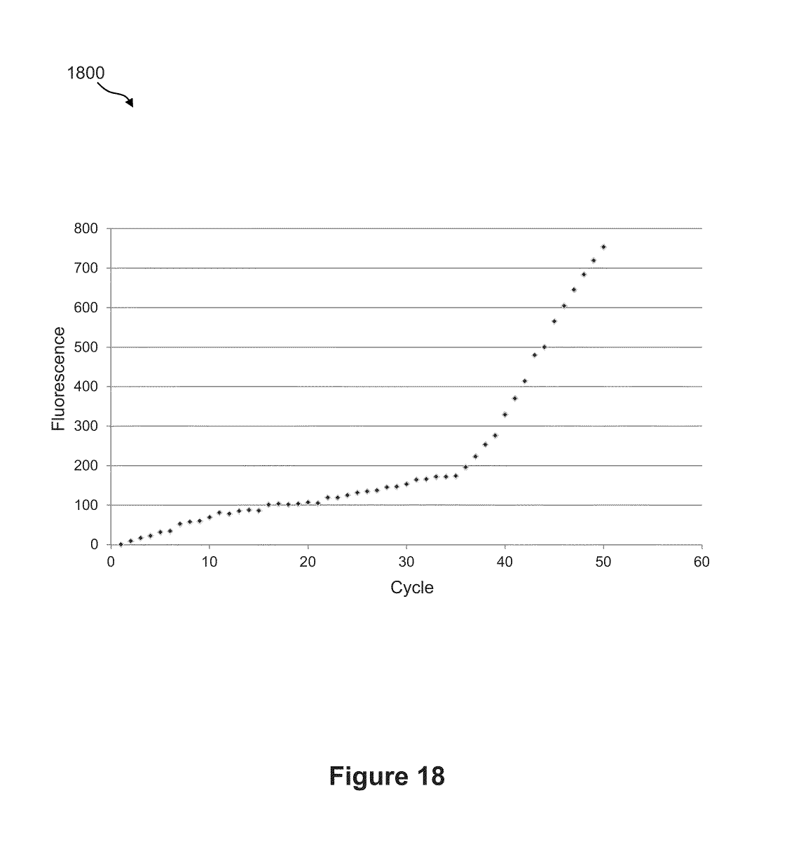

FIG. 18 shows a plot 1800, which is an example of a plot of fluorescence data from HIV RNA isolated from a whole blood sample and amplified on a droplet actuator. HIV RNA was isolated from a whole blood sample using a sample preparation protocol that included an agglutination step as described in reference to FIG. 17B. In this example, the agglutinate was erythroagglutinin PHA-E.

### 7.5 Systems

It will be appreciated that various aspects of the invention may be embodied as a method, system, computer readable medium, and/or computer program product. Aspects of the invention may take the form of hardware embodiments, software embodiments (including firmware, resident software, micro-code, etc.), or embodiments combining software and hardware aspects that may all generally be referred to herein as a “circuit,” “module” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Program code for carrying out operations of the invention may be written in an object oriented programming language such as Java, Smalltalk, C++ or the like. However, the program code for carrying out operations of the invention may also be written in conventional procedural programming languages, such as the “C” programming language or similar programming languages. The program code may be executed by a processor, application specific integrated circuit (ASIC), or other component that executes the program code. The program code may be simply referred to as a software application that is stored in memory (such as the computer readable medium discussed above). The program code may cause the processor (or any processor-controlled device) to produce a graphical user interface (“GUI”). The graphical user interface may be visually produced on a display device, yet the graphical user interface may also have audible features. The program code, however, may operate in any processor-controlled device, such as a computer, server, personal digital assistant, phone, television, or any processor-controlled device utilizing the processor and/or a digital signal processor.

The program code may locally and/or remotely execute. The program code, for example, may be entirely or partially stored in local memory of the processor-controlled device. The program code, however, may also be at least partially remotely stored, accessed, and downloaded to the processor-controlled device. A user's computer, for example, may entirely execute the program code or only partly execute the program code. The program code may be a stand-alone software package that is at least partly on the user's computer and/or partly executed on a remote computer or entirely on a remote computer or server. In the latter scenario, the remote computer may be connected to the user's computer through a communications network.

The invention may be applied regardless of networking environment. The communications network may be a cable network operating in the radio-frequency domain and/or the Internet Protocol (IP) domain. The communications network, however, may also include a distributed computing network, such as the Internet (sometimes alternatively known as the “World Wide Web”), an intranet, a local-area network (LAN), and/or a wide-area network (WAN). The communications network may include coaxial cables, copper wires, fiber optic lines, and/or hybrid-coaxial lines. The communications network may even include wireless portions utilizing any portion of the electromagnetic spectrum and any signaling standard (such as the IEEE 802 family of standards, GSM/CDMA/TDMA or any cellular standard, and/or the ISM band). The communications network may even include powerline portions, in which signals are communicated via electrical wiring. The invention may be applied to any wireless/wireline communications network, regardless of physical componentry, physical configuration, or communications standard(s).

Certain aspects of invention are described with reference to various methods and method steps. It will be understood that each method step can be implemented by the program code and/or by machine instructions. The program code and/or the machine instructions may create means for implementing the functions/acts specified in the methods.

The program code may also be stored in a computer-readable memory that can direct the processor, computer, or other programmable data processing apparatus to function in a particular manner, such that the program code stored in the computer-readable memory produce or transform an article of manufacture including instruction means which implement various aspects of the method steps.

The program code may also be loaded onto a computer or other programmable data processing apparatus to cause a series of operational steps to be performed to produce a processor/computer implemented process such that the program code provides steps for implementing various functions/acts specified in the methods of the invention.

### REFERENCES

CDC protocol of realtime RTPCR for influenza A (H1N1): World Health Organization; 30 Apr. 2009. REF #I-007-05.
Selvaraju S B, Selvarangan R. Evaluation of three influenza A and B real-time reverse transcription-PCR assays and a new 2009 H1N1 assay for detection of influenza viruses. J Clin Microbiol. November 2010; 48(11):3870-3875.
O'Connell K P, Bucher J R, Anderson P E, et al. Real-time fluorogenic reverse transcription-PCR assays for detection of bacteriophage MS2. Appl Environ Microbiol. January 2006; 72(1):478-483.

### CONCLUDING REMARKS

The foregoing detailed description of embodiments refers to the accompanying drawings, which illustrate specific embodiments of the invention. Other embodiments having different structures and operations do not depart from the scope of the present invention. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

We claim:

1. A method for multiplexed real-time amplification and detection of multiple target sequences in a single droplet on a droplet actuator, the method comprising:
a. loading a polymerase chain reaction (PCR) reaction mixture onto the droplet actuator;
b. dispensing a first PCR reaction droplet onto a droplet operations surface of the droplet actuator having droplet operations electrodes arranged thereon;
c. dispensing a second PCR reaction droplet onto the droplet operations surface of the droplet actuator, and combining the second PCR reaction droplet with the first PCR reaction droplet using droplet operations to form a combined PCR reaction droplet;
d. thermal cycling the combined PCR reaction droplet to form an amplified sample droplet; and
e. detecting fluorescence signals from the combined PCR reaction droplet during amplification.

2. The method of claim 1 wherein the first PCR reaction droplet comprises a 1×PCR reaction droplet, the second PCR reaction droplet comprises a 1×PCR reaction droplet, and the combined PCR reaction droplet comprises a 2×PCR reaction droplet.

3. The method of claim 1 wherein fluorescence signals from the amplified sample droplet are detected in real time during amplification.

4. The method of claim 1 wherein the PCR reaction mixture comprises sequence-specific DNA probes that are each labeled with a different fluorophore for detection of multiple target sequences in the single droplet.

5. The method of claim 4 wherein the PCR reaction mixture comprises four (4) sequence-specific DNA probes each labeled with four (4) different fluorophores.

6. The method of claim 4 wherein the multiple target sequences comprise an internal control sequence and one or more different target sequences.

7. The method of claim 1 wherein the amplified sample droplet following amplification is transported using droplet operations to a recovery reservoir on the droplet actuator.

8. The method of claim 1 wherein thermal cycling comprises multiple cycles of the combined PCR reaction droplet through a plurality of temperature control zones, wherein the temperature control zones comprise differing temperatures from one another.

9. The method of claim 1 wherein loading the PCR reaction mixture onto the droplet actuator comprises loading the PCR reaction mixture into a fluid dispensing reservoir of the droplet actuator

10. The method of claim 8 wherein a cycle of the combined PCR reaction droplet thermal cycling, comprises:
a. transporting the combined PCR reaction droplet using droplet operations to a first temperature control zone and incubating for a first period of time at a first temperature;
b. transporting the combined PCR reaction droplet using droplet operations from the first temperature control zone to a second temperature control zone and incubating for a second period of time at a second temperature; and
c. transporting the combined PCR reaction droplet using droplet operations from the second temperature control zone to a third temperature control zone and incubating for a third period of time at a third temperature.

11. The method of claim 10 wherein the combined PCR reaction droplet comprises DNA.

12. The method of claim 11 wherein the first period of time is sufficient for denaturation of DNA, the second period of time is sufficient for primer and probe annealing, and the third period of time is sufficient for primer elongation.

13. The method of claim 12 wherein the first period of time is in the range of about seven (7) seconds, the second period of time is in the range of about forty (40) seconds, and the third period of time is in the range of about less than one (1) second.

14. The method of claim 11 wherein the first temperature is sufficient for denaturation of DNA, the second temperature is sufficient for primer and probe annealing, and the third temperature is sufficient for primer elongation.

15. The method of claim 14 wherein the first temperature is in the range of about 95° C., the second temperature is in the range of about 55° C., and the third temperature is in the range of about 72° C.

16. The method of claim 12 wherein the first period of time sufficient for denaturation of DNA is determined using DNA melting analysis, comprising: labeling an amplified DNA sample with a fluorescent dye; loading the labeled amplified DNA sample onto the droplet actuator; dispensing and transporting the labeled amplified DNA sample using droplet operations to a detection spot within a temperature control zone on the droplet actuator heated to in the range of about 95° C.; measuring the fluorescence at regular time intervals until the fluorescence signal drops to a plateau level, wherein the time period to reach the plateau level correlates to an estimate of a required minimal PCR denaturation time.

17. The method of claim 12 wherein detecting fluorescence signals is conducted during the second period of time.

18. The method of claim 1 wherein detecting fluorescence signals, comprises:
a. transporting the combined PCR reaction droplet to a detection spot on the droplet actuator using droplet operations;
b. activating a detection system; and
c. measuring the fluorescence signals.

19. The method of claim 18 wherein the detection system comprises a four channel detection system.

20. The method of claim 18 wherein detecting fluorescence signals from the combined PCR reaction droplet occurs in real-time during amplification.

21. The method of claim 18 wherein the detection spot is smaller in size than droplet operations electrode upon which the combined PCR reaction droplet is transported to.

22. The method of claim 1 wherein the method is used for multiplexed detection of one or more types of influenza virus in a whole blood sample.

23. The method of claim 1 wherein the method is used for multiplexed detection of one or more types of human immunodeficiency virus (HIV) in a whole blood sample.

24. A method for multiplexed amplification and detection of multiple target sequences in a single droplet on a droplet actuator, the method comprising:
a. loading an on-bench prepared PCR reaction mixture onto the droplet actuator;
b. dispensing a droplet of the on-bench prepared PCR reaction mixture onto a droplet operations surface of the droplet actuator having droplet operations electrodes arranged thereon to form a sample droplet;
c. thermal cycling the sample droplet to form an amplified sample droplet; and
d. detecting fluorescence signals from the sample droplet during amplification.

25. A method of probe hybridization for detection of a target sequence in a single droplet on a droplet actuator, the method comprising:
a. amplifying a target sequence by PCR using a forward primer and a reverse primer;
b. locating one or more probe sites between the forward primer and the reverse primer;
c. providing a labeled probe designed to bind to a specific target sequence within the one or more probe sites; and
d. extending the forward primer by a polymerase enzyme and degrading the labeled probe to produce a detectable signal.

26. The method of claim 25 wherein exonuclease activity degrades the labeled probe.

27. The method of claim 25 wherein the detectable signal generated is proportional to a concentration of the target sequence.

28. The method of claim 25 wherein the labeled probe comprises a fluorescently-labeled probe.

29. The method of claim 25 wherein the labeled probe comprises an oligonucleotide probe that is labeled with a fluorescent reporter molecule and a quencher molecule

30. The method of claim 29 wherein the labeled probe comprises a FRET probe.

31. The method of claim 25 wherein the target sequence comprises a specific DNA sequence

32. The method of claim 30 wherein the FRET probe comprises a fluorophore covalently attached to a 5′-end of the oligonucleotide probe and a quencher molecule at a 3′-end.

33. The method of claim 32 wherein the FRET probe is degraded by exonuclease activity and the fluorophore is separated from the quencher producing a fluorescent signal.

34. A detection system for detecting multiple different signals in a single sample droplet at a single detection spot on a droplet actuator, comprising:
a. an arrangement of multiple excitation light-emitting diodes (LEDs); and
b. an arrangement of multiple detectors.

35. The detection system of claim 34 further comprising:
a. multiple excitation lenses aligned with the multiple excitation LEDs, and structured to focus light emitted from a corresponding LED onto a corresponding excitation filter;
b. multiple excitation filters aligned with the multiple excitation lenses, and structured each to select a different certain wavelength of light emitted from its corresponding LED for excitation of a certain signal;
c. multiple mirrors aligned with the multiple excitation filters, and structured to direct the filtered light to a corresponding second directing mirror that is positioned in proximity of a corresponding excitation focusing lens; and
wherein each directing mirror and corresponding focusing lens together multiplex the different wavelengths of light into a single excitation beam

36. The detection system of claim 34 further comprising:
a. multiple detection lenses aligned with the multiple detectors;
b. multiple detection filters aligned with the multiple detection lenses;
c. multiple dichroic filters aligned with the multiple detection filters, and structured to select a certain wavelength of light corresponding to an emission wavelength of a certain signal;
d. multiple focusing lens aligned with the multiple dichroic filters; and
wherein, the multiple focusing lens and directing mirrors together multiplex different wavelengths of light emitted from one or more signals from a single sample droplet into a single detection beam.

37. The detection system of claim 34 wherein the detection system comprises a single excitation beam and a single detection beam directed to the single detection spot on the droplet actuator.

38. The detection system of claim 34 wherein detection system is capable of detecting four (4) different signals.

39. The detection system of claim 34 wherein the multiple different signals comprise fluorescent signals.

40. The detection system of claim 34 wherein the detection system is enclosed in a housing.

41. The detection system of claim 40 wherein the housing is attached to a base plate adapted to position the detection system in a microfluidics system having the droplet actuator.

42. A droplet actuator for multiplexed real-time amplification and detection of multiple target sequences in a single droplet, comprising:
a. a bottom substrate separated from a top substrate to form a droplet operations gap, wherein the droplet operations gap is filled with a filler fluid;
b. one or more fluid reservoirs;
c. an electrode arrangement disposed on the bottom and/or top substrate comprising at least one of a path, line, and array of droplet operations electrodes; and
d. a plurality of temperature control zones, wherein the temperature control zones comprise differing temperatures from one another.

43. The droplet actuator of claim 42 wherein the one or more fluid reservoirs comprise at least one sample reservoir and one or more reagent reservoirs.

44. The droplet actuator of claim 43 wherein the at least one sample reservoir and the one or more reagent reservoirs each include an input port for loading fluids therein.

45. The droplet actuator of claim 42 wherein the droplet operations electrodes comprise electrowetting electrodes.

46. The droplet actuator of claim 43 wherein each of the one or more reagent reservoirs has a reagent dispensing electrode arranged with respect to the at least one of a path, line, and array of droplet operations electrodes.

47. The droplet actuator of claim 43 wherein each of the at least one sample reservoirs has a sample dispensing electrode, wherein the sample dispensing electrode is segmented into an arrangement of multiple individually controlled electrodes arranged with respect to the at least one of a path, line, and array of droplet operations electrodes.

48. The droplet actuator of claim 47 wherein the at least one sample reservoir is designed and structured to perform droplet operations comprising droplet mixing and/or droplet dispensing operations.

49. The droplet actuator of claim 42 further comprising one or more magnets positioned in proximity to certain of the droplet operations electrodes.

50. The droplet actuator of claim 49 wherein the one or more magnets are embedded within a deck that holds the droplet actuator.

51. The droplet actuator of claim 49 wherein the one or more magnets are positioned in a manner that ensures spatial immobilization of magnetically responsive beads during droplet dispensing operations.

52. The droplet actuator of claim 43 further comprising at least one of a sample dispensing region associated with the at least one sample reservoir and a droplet operations region associated with the one or more reagent reservoirs.

53. The droplet actuator of claim 52 wherein the height of the gap between the bottom substrate and the top substrate at each region may vary.

54. A system for performing multiplexed real-time amplification and detection of multiple target sequences in a single droplet on a droplet actuator, comprising a processor for executing code and a memory in communication with the processor, the system comprising code stored in the memory that causes the processor at least to:
a. load a polymerase chain reaction (PCR) reaction mixture onto the droplet actuator;
b. dispense a first PCR reaction droplet onto a droplet operations surface of the droplet actuator having droplet operations electrodes arranged thereon;
c. dispense a second PCR reaction droplet onto the droplet operations surface of the droplet actuator having droplet operations electrodes arranged thereon, and combining the second PCR reaction droplet with the first PCR reaction droplet using droplet operations to form a combined PCR reaction droplet;
d. thermal cycle the combined PCR reaction droplet to form an amplified sample droplet; and
e. detect fluorescence signals from the combined PCR reaction droplet during amplification.

55. A computer readable medium storing processor executable instructions for performing a method of performing multiplexed real-time amplification and detection of multiple target sequences in a single droplet on a droplet actuator, the method comprising:
a. loading a polymerase chain reaction (PCR) reaction mixture onto the droplet actuator;
b. dispensing a first PCR reaction droplet onto a droplet operations surface of the droplet actuator having droplet operations electrodes arranged thereon;
c. dispensing a second PCR reaction droplet onto the droplet operations surface of the droplet actuator having droplet operations electrodes arranged thereon, and combining the second PCR reaction droplet with the first PCR reaction droplet using droplet operations to form a combined PCR reaction droplet;
d. thermal cycling the combined PCR reaction droplet to form an amplified sample droplet; and
e. detecting fluorescence signals from the combined PCR reaction droplet during amplification.

56. A method for integrated sample preparation and multiplexed detection of infectious agents on a droplet actuator, the method comprising:
a. combining a whole blood sample with a volume of a lysis buffer solution in a sample reservoir of the droplet actuator;
b. incubating the combined whole blood sample and the volume of the lysis buffer solution for a period of time sufficient to yield a lysate comprising released nucleic acids;
c. adding magnetically responsive nucleic acid capture beads suspended in a binding buffer to the lysate;
d. incubating and mixing the magnetically responsive nucleic acid capture beads in the lysate for a period of time sufficient to allow binding of nucleic acids in the lysate to the magnetically responsive nucleic acid capture beads;
e. washing the magnetically responsive nucleic acid capture beads to remove unbound material to yield a washed bead-containing droplet substantially lacking unbound material;
f. repeating step (e) to produce a concentrated sample droplet comprising purified RNA bound to the magnetically responsive nucleic acid capture beads; and
g. transporting the concentrated sample droplet away from one or more magnets on the droplet actuator for further processing on the droplet actuator, wherein the further processing comprises a method for multiplexed detection of infectious agents in a single droplet on the droplet actuator.

57. The method of claim 56, wherein step (e) is repeated about 10 times.

58. The method of claim 56, wherein the method for multiplexed detection of infectious agents in a single droplet on the droplet actuator comprises execution of a droplet based RT-qPCR PCR amplification protocol.

59. The method of claim 56, wherein the multiplexed detection of infectious agents comprises detection of one or more types of influenza virus and/or human immunodeficiency virus (HIV).

60. A method for integrated sample preparation and multiplexed detection of infectious agents on a droplet actuator, the method comprising:
a. combining a whole blood sample with a volume of a lysis buffer solution in a sample reservoir of the droplet actuator;
b. incubating the combined whole blood sample and the volume of the lysis buffer solution for a period of time sufficient to yield a lysate comprising released nucleic acids;
c. adding magnetically responsive nucleic acid capture beads suspended in a binding buffer to the lysate;
d. incubating and mixing the magnetically responsive nucleic acid capture beads in the lysate for a period of time sufficient to allow binding of nucleic acids in the lysate to the magnetically responsive nucleic acid capture beads;
e. washing the magnetically responsive nucleic acid capture beads to remove unbound material to yield a washed bead-containing droplet substantially lacking unbound material;
f. repeating step (e) to produce a concentrated sample droplet comprising purified RNA;
g. eluting the purified RNA from the concentrated sample droplet with an elution buffer to produce eluted purified RNA;
h. re-binding the eluted purified RNA to the magnetically responsive nucleic acid capture beads;
i. repeating steps (e) through (g) to produce a droplet comprising eluted purified RNA; and
j. transporting the droplet comprising eluted purified RNA away from the magnetically responsive nucleic acid capture beads for further processing on the droplet actuator, wherein the further processing comprises a method for multiplexed detection of infectious agents in a single droplet on the droplet actuator.

61. The method of claim 60, wherein step (e) is repeated about 10 times.

62. The method of claim 60, wherein the method for multiplexed detection of infectious agents in a single droplet on the droplet actuator comprises execution of a droplet based RT-qPCR PCR amplification protocol.

63. The method of claim 60, wherein the multiplexed detection of infectious agents comprises detection of one or more types of influenza virus and/or human immunodeficiency virus (HIV).

64. A method for integrated sample preparation and multiplexed detection of infectious agents on a droplet actuator, the method comprising:
a. combining a whole blood sample with a volume of a lysis buffer solution and a volume of an agglutinate in a sample reservoir of the droplet actuator;
b. incubating the combined whole blood sample, the volume of the lysis buffer solution, and the agglutinate for a period of time sufficient for agglutination and to yield a lysate comprising released nucleic acids;
c. adding magnetically responsive nucleic acid capture beads suspended in a binding buffer to the lysate;
d. incubating and mixing the magnetically responsive nucleic acid capture beads in the lysate for a period of time sufficient to allow binding of nucleic acids in the lysate to the magnetically responsive nucleic acid capture beads;
e. concentrating the magnetically responsive nucleic acid capture beads and dispensing a concentrated sample droplet comprising the magnetically responsive nucleic acid capture beads onto a droplet operations electrode, wherein the droplet operations electrode is between flanking electrodes and near at least one magnet;
f. washing the magnetically responsive nucleic acid capture beads to remove unbound material to yield a washed bead-containing droplet substantially lacking unbound material;
g. repeating step (f) to produce a concentrated sample droplet comprising purified RNA bound to the magnetically responsive nucleic acid capture beads;
h. transporting the concentrated sample droplet away from one or more magnets on the droplet actuator for further processing on the droplet actuator, wherein the further processing comprises a method for multiplexed detection of infectious agents in a single droplet on the droplet actuator.

65. The method of claim 64, wherein the agglutinate is erythroagglutinin PHA-E.

66. The method of claim 64, wherein prior to step (e), the droplet operations electrode is flooded with wash buffer to facilitate dilution and microfluidic operations.

67. The method of claim 64, wherein step (f) is repeated from about 10 times to about 30 times.

68. The method of claim 64, wherein the method for multiplexed detection of infectious agents in a single droplet on the droplet actuator comprises execution of a droplet based RT-qPCR PCR amplification protocol.

69. The method of claim 64, wherein the multiplexed detection of infectious agents comprises detection of one or more types of influenza virus and/or human immunodeficiency virus (HIV).

70. A method for integrated sample preparation and multiplexed detection of infectious agents on a droplet actuator, the method comprising:
a. combining a whole blood sample with a volume of a lysis buffer solution and a volume of an agglutinate in a sample reservoir of the droplet actuator;
b. incubating the combined whole blood sample, the volume of the lysis buffer solution, and the agglutinate for a period of time sufficient for agglutination and to yield a lysate comprising released nucleic acids;
c. adding magnetically responsive nucleic acid capture beads suspended in a binding buffer to the lysate;
d. incubating and mixing the magnetically responsive nucleic acid capture beads in the lysate for a period of time sufficient to allow binding of nucleic acids in the lysate to the magnetically responsive nucleic acid capture beads;
e. concentrating the magnetically responsive nucleic acid capture beads and dispensing a concentrated sample droplet comprising the magnetically responsive nucleic acid capture beads onto a droplet operations electrode, wherein the droplet operations electrode is between flanking electrodes and near at least one magnet;
f. washing the magnetically responsive nucleic acid capture beads in the concentrated sample droplet to remove unbound material to yield a washed bead-containing droplet substantially lacking unbound material;
g. repeating step (f) to produce a concentrated sample droplet comprising purified RNA;
h. eluting the purified RNA from the concentrated sample droplet with an elution buffer to produce eluted purified RNA; and
i. transporting the eluted purified RNA away from the magnetically responsive nucleic acid capture beads for further processing on the droplet actuator, wherein the further processing comprises a method for multiplexed detection of infectious agents in a single droplet on the droplet actuator.

71. The method of claim 70, wherein the agglutinate is erythroagglutinin PHA-E.

72. The method of claim 70, wherein prior to step (e), the droplet operations electrode is flooded with wash buffer to facilitate dilution and microfluidic operations.

73. The method of claim 70, wherein step (f) is repeated from about 10 times to about 30 times.

74. The method of claim 70, wherein the method for multiplexed detection of infectious agents in a single droplet on the droplet actuator comprises execution of a droplet based RT-qPCR PCR amplification protocol.

75. The method of claim 70, wherein the multiplexed detection of infectious agents comprises detection of one or more types of influenza virus and/or human immunodeficiency virus (HIV).


SEQUENCE LISTING (15 sequences)

SEQ ID NO: 1 - DNA, 20 bp, Artificial Sequence
  PCR forward primer
atccgggaagtgttcttcat

SEQ ID NO: 2 - DNA, 20 bp, Artificial Sequence
  PCR reverse primer
accagcgcgctggtagatga

SEQ ID NO: 3 - DNA, 20 bp, Artificial Sequence
  PCR forward primer
tcatctaccagcgcgctgtt

SEQ ID NO: 4 - DNA, 20 bp, Artificial Sequence
  PCR reverse primer
atgaagaacacttcccggat

SEQ ID NO: 5 - DNA, 20 bp, Artificial Sequence
  PCR forward primer
ggtgcggatcagctcgagaa

SEQ ID NO: 6 - DNA, 21 bp, Artificial Sequence
  PCR reverse primer
cgacgtcggacgcagtctttt

SEQ ID NO: 7 - DNA, 22 bp, Artificial Sequence
  PCR forward primer
gaccratcctgtcacctctgac

SEQ ID NO: 8 - DNA, 24 bp, Artificial Sequence
  PCR reverse primer
agggcattytggacaaakcgtcta

SEQ ID NO: 9 - DNA, 24 bp, Artificial Sequence
  DNA probe
tgcagtcctcgctcactgggcacg

SEQ ID NO: 10 - DNA, 22 bp, Artificial Sequence
  PCR forward primer
tcctcaactcactcttcgagcg

SEQ ID NO: 11 - DNA, 21 bp, Artificial Sequence
  PCR reverse primer
cggtgctcttgaccaaattgg

SEQ ID NO: 12 - DNA, 27 bp, Artificial Sequence
  DNA probe
ccaattcgagcagctgaaactgcggtg

SEQ ID NO: 13 - DNA, 21 bp, Artificial Sequence
  PCR forward primer
gctctgagagcggctctattg

SEQ ID NO: 14 - DNA, 18 bp, Artificial Sequence
  PCR reverse primer
cgttatagcggaccgcgt

SEQ ID NO: 15 - DNA, 21 bp, Artificial Sequence
  DNA probe
ccgagaccaatgtgcgccgtg